SEQUENCE LISTING (1 sequence)

SEQ ID NO: 1 - protein, 33 aa, ARTIFICIAL
  SITE 1..1: This residue is L-His, imidazopropionyl, alpha-hydroxy-His, D-His, deamino-His, 2-amino-His, beta-hydroxy-His, homo-His, N-alpha-acetyl-His, N-alpha-formyl-His, alpha-fluoromethyl-His, alpha-methyl-His, 3-pyridyl-Ala, 2-pyridyl-Ala, or 4-pyridyl-Ala
  Based on human GLP-1
  SITE 2..2: This residue is Ala, Gly, Val, Leu, Ile, Thr, Ser, Lys, alpha-aminoisobutyric acid, (1-aminocyclopropyl)carboxylic acid, (1-aminocyclobutyl)carboxylic acid, (1-aminocyclopentyl)carboxylic acid, (1-aminocyclohexyl)carboxylic acidcarboxylic acid, or (1-aminocycloheptyl)carboxylic acid
  SITE 6..6: This residue is Lys or Phe
  SITE 10..10: This residue is Val or Leu
  SITE 12..12: This residue is Ser, Arg, Asn, Gln, or Glu
  SITE 13..13: This residue is Tyr or Gln
  SITE 14..14: This residue is Leu, Lys, or Met
  SITE 16..16: This residue is Gly, Glu, Lys, or alpha- aminoisobutyric acid
  SITE 17..17: This residue is Gln, Glu, or Arg
  SITE 18..18: This residue is Ala or Lys
  SITE 19..19: This residue is Ala or Val
  SITE 20..20: This residue is Val, His, Lys or Arg
  SITE 24..24: This residue is Ala, Glu, or Arg
  SITE 25..25: This residue is Trp or His
  SITE 28..28: This residue is Glu, Asn, Gly, Gln, or Arg
  SITE 29..29: This residue is Gly, alpha-aminoisobutyric acid, or absent
  SITE 30..30: This residue is Arg, Gly, Lys, or absent
  SITE 31..31: This residue is Gly, Ala, Glu, Pro, Lys, Arg, or absent
  SITE 32..32: This residue is Ser, Gly, Ala, Glu, Gln, Pro, Arg, or absent
  SITE 33..33: This residue is Gly or absent
XXEGTXTSDXSXXXEXXXXXKFIXXLVXXXXXX

(19) United States
(12) Patent Application Publication
(10) Pub. No.: US 2024/0041983 A1
(43) Pub. Date: Feb. 8, 2024

(54) IMPROVED PHARMACEUTICAL FORMULATIONS OF GLP-1 RECEPTOR AGONISTS

(71) Applicant: CYPRUMED GMBH, Obsteig (AT)

(72) Inventors: Florian FÖGER, Innsbruck (AT); Martin WERLE, Bludenz (AT)

(21) Appl. No.: 18/043,976

(22) PCT Filed: Sep. 7, 2021
(86) PCT No.: PCT/EP2021/074625

§ 371 Date: Mar. 3, 2023

(30) Foreign Application Priority Data
Sep. 7, 2020 (EP) 20194828.8
Nov. 19, 2020 (EP) 20208628.6

(51) Int. Cl. A61K 38/26; A61K 9/48; A61K 9/28; A61K 9/20

(52) U.S. Cl. CPC A61K 38/26; A61K 9/4891; A61K 9/4816; A61K 9/2846; A61K 9/2013

(57) ABSTRACT

The present invention relates to a solid oral pharmaceutical composition comprising (i) a core comprising a GLP-1 receptor agonist, and (ii) a first coating, wherein the first coating comprises a copolymer (A) in combination with a copolymer (B) and/or a copolymer (C) and/or a copolymer (D).

The present invention relates to a solid oral pharmaceutical composition comprising (i) a core comprising a Glucagon-Like-Peptide-1 (GLP-1) receptor agonist, and (ii) a first coating, wherein the first coating comprises a copolymer (A) in combination with a copolymer (B) and/or a copolymer (C) and/or a copolymer (D).

Glucagon-like peptide-1 (GLP-1) is a 30-amino acid incretin peptide hormone, which is secreted by enteroendocrine L cells in the gastrointestinal tract (GIT) and by preproglucagon neurons located in the nucleus of the solitary tract in the hindbrain. Pharmacologically, long-acting GLP-1 receptor agonists (GLP-1RAs) exhibit glucoregulatory functions via a triumvirate of mechanisms, namely, stimulation of insulin release in a glucose-dependent manner, suppression of glucagon activity during hyperglycemia, and a minor delay of gastric emptying resulting in slower glucose absorption. In addition, GLP-1 promotes satiety and reduces energy intake by virtue of its neurotransmitter role in brainstem-hypothalamus pathways signaling satiety, and some long-acting GLP-1RAs including semaglutide have shown cardiovascular risk reduction.

Recent advancements in fatty acid acylation-based protraction technology have provided the possibility of achieving extended plasma half-lives without increasing molecular size, leading to the discovery of semaglutide, a GLP-1 analog with a t½ of ˜1 week in humans (Lau, J. et al., J Med Chem 2015, 58, 7370-7380).

Despite the remarkable pharmacological effects of simultaneous glucose, body weight, and blood pressure lowering and cardiovascular risk reduction already achieved with semaglutide administered subcutaneously, the mode of administration is probably a barrier for some potential users. This barrier could be overcome with the availability of an oral formulation of semaglutide or other GLP-1 agonists. Conceivably, an orally administered GLP-1RA may lead to earlier initiation of GLP-1RA treatment and improve acceptance and adherence among patients, compared with injectable formulations of GLP-1RAs. The inherent physicochemical properties of peptides, such as high molecular weight, enzymatical lability, hydrophilicity, and low permeability, have hampered attempts to deliver peptides such as GLP-1 via the oral route (Aguirre, T. A. et al., Adv Drug Deliv Rev 2016, 106, 223-241). The main difficulties lie in surmounting the challenges presented by the hostile environment of the GIT, which is designed to degrade proteins and peptides ingested in food to di- and tripeptides before absorption in the small intestine.

A solid dosage form facilitating the oral administration of GLP-1 receptor agonists overcoming these challenges is thus highly desirable. Advantages of solid oral dosage forms over other dosage forms generally include ease of manufacture, storage and administration. There are also advantages relating to convenience of administration increasing patient compliance. The oral administration of GLP-1 receptor agonists is however very challenging due to their poor bioavailability and the issues set out above. Indeed, only one oral formulation of a GLP-1 receptor agonist has received regulatory approval to date (i.e., Rybelsus® containing the GLP-1 agonist semaglutide), despite having an oral bioavailability of only about 1%, which attests to the difficulty of providing formulations of GLP-1 receptor agonists that are suitable for oral administration. With respect to Rybelsus® it is furthermore noted that it must be taken on an empty stomach (after overnight fasting) followed by additional 30 minutes without food intake or high water intake.

The present invention addresses these shortcomings in the art and provides solid oral pharmaceutical compositions of GLP-1 receptor agonists, which are particularly well suited for oral administration due to their advantageous release profile, improved bioavailability and reduced food effects.

Accordingly, the present invention provides a solid oral pharmaceutical composition comprising:
(i) a core comprising a GLP-1 receptor agonist, and
(ii) a first coating, wherein the first coating comprises
   (ii-1) a copolymer (A) in combination with
   (ii-2) a copolymer (B) and/or a copolymer (C) and/or a copolymer (D);
   wherein the copolymer (A) comprises:
      (a) 20 to 90 mol-% ethyl acrylate repeating units, and
      (b) 10 to 80 mol-% methyl methacrylate repeating units;
   wherein the copolymer (B), if present, comprises:
      (a) 25 to 75 mol-% methacrylic acid repeating units, and
      (b) 25 to 75 mol-% ethyl acrylate repeating units;
   wherein the copolymer (C), if present, comprises:
      (a) 25 to 60 mol-% methacrylic acid repeating units, and
      (b) 40 to 75 mol-% methyl methacrylate repeating units;
   wherein the copolymer (D), if present, comprises:
      (a) 5 to 20 mol-% methacrylic acid repeating units, and
      (b) 20 to 40 mol-% methyl methacrylate repeating units, and
      (c) 60 to 75 mol-% methyl acrylate repeating units.

In the context of the present invention, it was surprisingly found that the coating of the solid oral pharmaceutical composition provided herein results in an advantageous release profile significantly below pH 7 upon oral administration, allowing the release of the GLP-1 receptor agonist in the distal small intestine, as well as an improved bioavailability as compared to conventional formulations of GLP-1 receptor agonists, including formulations with pH dependent enteric coatings. As also shown in the experimental examples, the pharmaceutical composition according to the invention has been found to allow dissolution at a comparatively low pH between 5.5 and 6.5. The release of the GLP-1 receptor agonist in the distal small intestine (distal jejunum or ileum) is advantageous in view of the reduced activity of proteolytic enzymes as compared to the proximal small intestine (duodenum and jejunum), in view of the reduced intestinal motility in this segment (leading to reduced dilution effects of the dissolving pharmaceutical composition, enabling a high concentration of the GLP-1 receptor agonist to achieve optimal absorption), and in view of the higher solubility of peptidic GLP-1 receptor agonists at the pH levels present in the distal jejunum or ileum as compared to those present in the stomach, duodenum or proximal jejunum. In addition, it has been found that the solid oral pharmaceutical composition of the present invention exhibits advantageously reduced negative food effects. This stands in contrast to known formulations of GLP-1 receptor agonists that target the proximal gastrointestinal tract, in particular those targeting and dissolving in the proximal GI-tract such as the stomach or duodenum having no coating or a coating consisting only of anionic polymers, where detrimental food interactions have been observed (Maarbjerg S J et al., Diabetes, 2017, 66: A321 (without coating), as well as Example 33 of WO 2016/120378 A1 (coating based on Eudragit FS 30 D)). The solid oral pharmaceutical composition of the present invention can thus deliver the GLP-1 receptor agonist with an improved independence from food intake by the subject to be treated.

The present invention further provides a solid oral pharmaceutical composition (as described above) for use in therapy, particularly for use in the treatment or prevention of diabetes, obesity, non-alcoholic fatty liver disease, non-alcoholic steatohepatitis (NASH), a cardiovascular disease or any other disease/disorder involving GLP-1.

The invention likewise relates to the use of a solid oral pharmaceutical composition (as described above) for the manufacture of a medicament for the treatment or prevention of diabetes, obesity, non-alcoholic fatty liver disease, non-alcoholic steatohepatitis (NASH), a cardiovascular disease or any other disease/disorder involving GLP-1.

Moreover, the invention refers to a method of treating or preventing a disease/disorder in a subject, wherein the disease/disorder is diabetes, obesity, non-alcoholic fatty liver disease, non-alcoholic steatohepatitis (NASH), a cardiovascular disease or any other disease/disorder involving GLP-1, the method comprising orally administering the solid oral pharmaceutical composition (as described above) to a subject in need thereof. It will be understood that a therapeutically effective amount should be administered in accordance with this method.

The invention also provides a method of orally delivering a GLP-1 receptor agonist, the method comprising orally administering the solid oral pharmaceutical composition (as described above).

As explained above, the present invention relates to the treatment or prevention of diabetes, obesity, non-alcoholic fatty liver disease, non-alcoholic steatohepatitis (NASH), a cardiovascular disease or any other disease/disorder involving GLP-1 (including any disease/disorder involving, or mediated by, a deficiency of GLP-1 or deficient GLP-1 signalling), using the solid oral pharmaceutical composition provided herein. The cardiovascular disease may be, e.g., atherosclerosis, myocardial infarction, coronary heart disease, stroke, heart insufficiency, heart failure (e.g., acute heart failure or chronic heart failure), coronary artery disease, hypertension, cardiomyopathy, reperfusion injury, cerebral ischemia, left ventricular hypertrophy, arrhythmia, cardiac dysrhythmia, syncope, angina pectoris, stenosis, or restenosis. The solid oral pharmaceutical composition can thus also be used for cardiovascular risk reduction. Among the aforementioned therapeutic indications, the treatment or prevention of diabetes is particularly preferred (such as, e.g., type 1 diabetes, type 2 diabetes, or gestational diabetes; or severe autoimmune diabetes, severe insulin-deficient diabetes, severe insulin-resistant diabetes, mild obesity-related diabetes, or mild age-related diabetes). Even more preferred is the treatment or prevention of type 2 diabetes.

It is preferred that the first coating dissolves at a pH in the range of 5 to 7, preferably at a pH in the range of 5.5 to 6.5, even more preferably at a pH in the range of 5.5 to 6.0. The components and optional components of the first coating, such as copolymers (A), (B), (C) and (D), will be described in the following.

Copolymer (A)

Copolymer (A), which is present in the first coating, comprises (a) 20 to 90 mol-% ethyl acrylate repeating units, and (b) 10 to 80 mol-% methyl methacrylate repeating units. The copolymer (A) preferably is a neutral copolymer or a cationic copolymer.

It is particularly preferred that the copolymer (A) in the first coating is a neutral non-ionic copolymer. Preferably at least 90 mol-%, more preferably at least 95 mol-%, even more preferably at least 98 mol-%, of the repeating units in copolymer (A) are selected from ethyl acrylate repeating units and methyl methacrylate repeating units. In particular, the copolymer (A) may consist of ethyl acrylate repeating units and methyl methacrylate repeating units. The copolymer (A) in the first coating preferably comprises 50 to 80 mol-% ethyl acrylate repeating units and 20 to 50 mol-% methyl methacrylate repeating units, more preferably 60 to 75 mol-% ethyl acrylate repeating units and 25 to 40 mol-% methyl methacrylate repeating units, even more preferably 64 to 68 mol-% ethyl acrylate repeating units and 32 to 36 mol-% methyl methacrylate repeating units. The molar ratio of ethyl acrylate repeating units to methyl methacrylate repeating units in the copolymer (A) is preferably from 1.5:1 to 2.5:1, more preferably from 1.8:1 to 2.2:1, even more preferably 2:1. A corresponding preferred example of copolymer (A) is poly(ethyl acrylate-co-methyl methacrylate) 2:1, particularly Eudragit NM 30 D, Eudragit NE 30 D, or Eudragit NE 40 D.

As explained above, the copolymer (A) in the first coating may also be a cationic copolymer. Accordingly, the copolymer (A) in the first coating may further comprise 0.5 to 20 mol-%, preferably 1 to 15 mol-%, of 2-(trimethylammonio)ethyl methacrylate chloride repeating units (in addition to the ethyl acrylate repeating units and the methyl methacrylate repeating units). For example, in a preferred composition, the copolymer (A) in the first coating comprises 25 to 39 mol-% ethyl acrylate repeating units, 60 to 74 mol-% methyl methacrylate repeating units, and 1 to 15 mol-% 2-(trimethylammonio)ethyl methacrylate chloride repeating units. In the case that copolymer (A) is a cationic copolymer, it is preferred that at least 90 mol-%, more preferably at least 95 mol-%, even more preferably at least 98 mol-%, of the repeating units in copolymer (A) are selected from ethyl acrylate repeating units, methyl methacrylate repeating units, and 2-(trimethylammonio)ethyl methacrylate chloride repeating units. In particular, copolymer (A) in the first coating may consist of ethyl acrylate repeating units, methyl methacrylate repeating units, and 2-(trimethylammonio)ethyl methacrylate chloride repeating units. The copolymer (A) in the first coating may comprise ethyl acrylate repeating units, methyl methacrylate repeating units, and 2-(trimethylammonio)ethyl methacrylate chloride repeating units, e.g., in a molar ratio of 1:2:0.1 or 1:2:0.2. Corresponding preferred examples of copolymer (A) are poly(ethyl acrylate-co-methyl methacrylate-co-2-(trimethylammonio)ethyl methacrylate chloride) 1:2:0.2, particularly Eudragit RL 30 D, or poly(ethyl acrylate-co-methyl methacrylate-co-2-(trimethylammonio)ethyl methacrylate chloride) 1:2:0.1, particularly Eudragit RS 30 D.

It is generally preferred that the copolymer (A) in the first coating does not comprise methyl acrylate repeating units. Thus, the copolymer (A) in the first coating preferably comprises not more than 3 mol-% methyl acrylate repeating units, more preferably not more than 1 mol-%, even more preferably not more than 0.5 mol-%, yet more preferably not more than 0.1 mol-%, still more preferably not more than 0.01 mol-%, most preferably 0 mol-% methyl acrylate repeating units.

It is preferred that the copolymer (A) in the first coating is obtained from an aqueous dispersion of copolymer (A).

The first coating may further comprise, in addition to the copolymer (A), one or more polymers selected from ethyl cellulose, hydroxypropyl methylcellulose (HPMC), and polyvinyl acetate. Moreover, the present invention in a further embodiment also relates to a solid oral pharmaceutical composition as described and defined herein, wherein the first coating comprises one or more polymers selected from ethyl cellulose, hydroxypropyl methylcellulose (HPMC), and polyvinyl acetate instead of the copolymer (A).

Copolymer (B)

The copolymer (B), if present in the first coating, comprises (a) 25 to 75 mol-% methacrylic acid repeating units and (b) 25 to 75 mol-% ethyl acrylate repeating units, preferably 45 to 55 mol-% methacrylic acid repeating units and 45 to 55 mol-% ethyl acrylate repeating units. The copolymer (B) preferably is an anionic copolymer. For example, the copolymer (B) in the first coating may comprise methacrylic acid repeating units and ethyl acrylate repeating units in a molar ratio of 0.5:1 to 1:0.5, preferably in a molar ratio of 0.8:1 to 1:08, more preferably in a molar ratio of 1:1. Preferably at least 90 mol-%, more preferably at least 95 mol-%, even more preferably at least 98 mol-%, of the repeating units in copolymer (B) are selected from methacrylic acid repeating units and ethyl acrylate repeating units. It is furthermore preferred that the copolymer (B) in the first coating consists of methacrylic acid repeating units and ethyl acrylate repeating units. A corresponding preferred example of copolymer (B) is poly(methacrylic acid-co-ethyl acrylate) 1:1, particularly Eudragit L 30 D-55 or Eudragit L 100 D-55.

It is generally preferred that the copolymer (B), if present in the first coating, does not comprise methyl acrylate repeating units. Thus, the copolymer (B) in the first coating preferably comprises not more than 3 mol-% methyl acrylate repeating units, more preferably not more than 1 mol-%, even more preferably not more than 0.5 mol-%, yet even more preferably not more than 0.1 mol-%, still more preferably not more than 0.01 mol-%, most preferably 0 mol-% methyl acrylate repeating units.

It is preferred that the copolymer (B) in the first coating is obtained from an aqueous dispersion of copolymer (B).

Copolymer (C)

The copolymer (C), if present in the first coating, comprises (a) 25 to 60 mol-% methacrylic acid repeating units and (b) 40 to 75 mol-% methyl methacrylate repeating units. The copolymer (C) preferably is an anionic copolymer. Preferably at least 90 mol-%, more preferably at least 95 mol-%, even more preferably at least 98 mol-%, of the repeating units in copolymer (C) are selected from methacrylic acid repeating units and methyl methacrylate repeating units. In particular, the copolymer (C) in the first coating may consist of methacrylic acid repeating units and methyl methacrylate repeating units. The copolymer (C) in the first coating preferably is a copolymer (C-1) or a copolymer (C-2), as described in the following. Accordingly, the first coating may comprise a copolymer (C-1), a copolymer (C-2), or the combination of both a copolymer (C-1) and a copolymer (C-2).

The copolymer (C-1) in the first coating comprises 25 to 60 mol-% methacrylic acid repeating units and 40 to 75 mol-% methyl methacrylate repeating units, preferably 45 to 55 mol-% methacrylic acid repeating units and 45 to 55 mol-% methyl methacrylate repeating units. Preferably at least 90 mol-%, more preferably at least 95 mol-%, even more preferably at least 98 mol-%, of the repeating units in copolymer (C-1) are selected from methacrylic acid repeating units and methyl methacrylate repeating units. For example, the copolymer (C-1) in the first coating may comprise methacrylic acid repeating units and methyl methacrylate repeating units in a molar ratio of 0.5:1 to 1.5:1, preferably in a molar ratio of 0.8:1 to 1:08, more preferably in a molar ratio of 1:1. In particular, the copolymer (C-1) in the first coating may consist of methacrylic acid repeating units and methyl methacrylate repeating units. A corresponding preferred example of copolymer (C-1) is poly(methacrylic acid-co-methyl methacrylate) 1:1, particularly Eudragit L 100 or Eudragit L 12.5.

The copolymer (C-2) in the first coating comprises 25 to 60 mol-% methacrylic acid repeating units and 40 to 75 mol-% methyl methacrylate repeating units, preferably 25 to 40 mol-% methacrylic acid repeating units and 60 to 75 mol-% methyl methacrylate repeating units. Accordingly, it is preferred that the copolymer (C-2) in the first coating comprises methacrylic acid repeating units and methyl methacrylate repeating units in a molar ratio of 1:1.5 to 1:2.5, more preferably in a molar ratio of 1:2. Preferably at least 90 mol-%, more preferably at least 95 mol-%, even more preferably at least 98 mol-%, of the repeating units in copolymer (C-2) are selected from methacrylic acid repeating units and methyl methacrylate repeating units. In particular, the copolymer (C-2) in the first coating may consist of methacrylic acid repeating units and methyl methacrylate repeating units. A corresponding preferred example of copolymer (C-2) is poly(methacrylic acid-co-methyl methacrylate) 1:2, particularly Eudragit S 100.

It is generally preferred that the copolymer (C), if present in the first coating, including copolymer (C-1) and/or copolymer (C-2), does not comprise methyl acrylate repeating units. Thus, the copolymer (C) in the first coating (or the copolymer (C-1) and/or the copolymer (C-2)) preferably comprises not more than 3 mol-% methyl acrylate repeating units, more preferably not more than 1 mol-%, even more preferably not more than 0.5 mol-%, yet even more preferably not more than 0.1 mol-%, still more preferably not more than 0.01 mol-%, most preferably 0 mol-% methyl acrylate repeating units.

It is preferred that the copolymer (C) in the first coating, including the copolymer (C-1) and/or the copolymer (C-2), is obtained from an aqueous dispersion of the respective copolymer (i.e., copolymer (C), copolymer (C-1) or copolymer (C-2)).

Copolymer (D)

The copolymer (D), if present in the first coating, comprises (a) 5 to 20 mol-% methacrylic acid repeating units, (b) 20 to 40 mol-% methyl methacrylate repeating units, and (c) 60 to 75 mol-% methyl acrylate repeating units. Preferably at least 90 mol-%, more preferably at least 95 mol-%, even more preferably at least 98 mol-%, of the repeating units in copolymer (D) are selected from methacrylic acid repeating units, methyl methacrylate repeating units, and methyl acrylate repeating units.

It is preferred that copolymer (D), if present in the first coating, comprises 7 to 13 mol-% methacrylic acid repeating units, 25 to 31 mol-% methyl methacrylate repeating units, and 62 to 68 mol-% methyl acrylate repeating units. The copolymer (D) in the first coating preferably comprises methacrylic acid repeating units, methyl methacrylate repeating units, and methyl acrylate repeating units in a molar ratio of 1:3:7. It is furthermore preferred that the copolymer (D), if present in the first coating, consists of methacrylic acid repeating units, methyl methacrylate repeating units, and methyl acrylate repeating units.

While the first coating may optionally comprise a copolymer (D), it is preferred that the first coating does not contain any copolymer (D).

Contents of the Respective Copolymers in the First Coating

The content of the copolymer (A) in the first coating is preferably at least 25% (w/w), more preferably at least 50% (w/w), even more preferably at least 75% (w/w), yet even more preferably at least 80% (w/w), still more preferably at least 90% (w/w), in relation to the total weight of the first coating.

As explained above, the first coating comprises the copolymer (A) in combination with the copolymer (B) and/or the copolymer (C) and/or the copolymer (D). It is preferred that the first coating comprises the copolymer (A) in combination with the copolymer (B) and/or the copolymer (C). It is more preferred that the first coating comprises the copolymer (A) and the copolymer (B). In this case, the content of the copolymer (A) in the first coating is preferably at least 25% (w/w), more preferably at least 50% (w/w), even more preferably at least 75% (w/w), yet even more preferably at least 80% (w/w), still more preferably at least 90% (w/w), in relation to the total weight of the copolymer (A) and the copolymer (B) in the first coating.

Alternatively, the first coating may also comprise the copolymer (A) and the copolymer (C). As explained above, the copolymer (C) is preferably a copolymer (C-1) or a copolymer (C-2). Accordingly, the first coating may comprise copolymer (A) and copolymer (C-1), or the first coating may comprise copolymer (A) and copolymer (C-2), or the first coating may comprise copolymer (A), copolymer (C-1) and copolymer (C-2).

It is to be understood that the first coating may further comprise one or more other polymers, particularly one or more polymers selected from ethyl cellulose, hydroxypropyl methylcellulose (HPMC), and polyvinyl acetate.

The first coating may, for example, account for at least 2% w/w, preferably 2 to 25% w/w, more preferably 3 to 20% w/w, even more preferably 3 to 15% w/w, in relation to the total weight of the solid oral pharmaceutical composition.

The first coating may further comprise one or more plasticizers. The one or more plasticizers are preferably selected from mono-, di- and tri-alkyl citrates such as, e.g., triethyl citrate, tripropyl citrate, tributyl citrate, or acetyl triethyl citrate; dialkyl sebacinates such as, e.g., diethyl sebacinate, dipropyl sebacinate, or dibutyl sebacinate; dialkyl phthalates such as, e.g., dimethyl phthalate, diethyl phthalate, dipropyl phthalate, dibutyl phthalate, or dioctyl phthalate; glycerol and mono-, di- and tri-glycerides such as, e.g., glyceryl triacetate, glyceryl tributyrate, glyceryl monostearate, or acetylated monoglycerides; propylene glycols and polyethylene glycols such as, e.g., PEG 300, PEG 400, PEG 600, PEG 800, PEG 1450, or PEG3350; fatty acids such as, e.g., stearic acid, oleic acid, or esters of fatty acids. More preferably, the one or more plasticizers are selected from mono-, di- and tri-alkyl citrates such as, e.g., triethyl citrate, tripropyl citrate, tributyl citrate or acetyl triethyl citrate. Even more preferably, the first coating further comprises 10 to 80% by weight, preferably 40 to 80% by weight, of one or more selected from triethyl citrate, tripropyl citrate and tributyl citrate, based on the total weight of the first coating. An example of a preferred plasticizer is PlasACRYL, such as PlasACRYL™ HTP20 and PlasACRYL™ T20.

It is preferred that the first coating is obtained from an aqueous dispersion of copolymer (A) and the copolymer (B) and/or copolymer (C) and/or the copolymer (D), optionally further containing any of the optional components of the first coating.

It will be understood that the first coating is exterior to the core which is comprised in the solid oral pharmaceutical composition. The first coating preferably surrounds (or completely covers) and contains the core. While the pharmaceutical composition may also contain one or more intermediate coatings in between the core and the first coating, as described herein below, it is preferred that there is no such intermediate layer, i.e. the first coating preferably is directly exterior to the core (or is in direct contact to the core).

Optional Second Coating

The solid oral pharmaceutical composition according to the present invention may comprise further coatings (in addition to the above-described first coating). In particular, the solid oral pharmaceutical composition preferably comprises a second coating which is exterior to the first coating, wherein the second coating comprises a copolymer (C). The second coating preferably surrounds (or completely covers) and contains the first coating. Furthermore, the second coating preferably dissolves at a pH in the range of 5 to 7, preferably at a pH in the range of 5.5 to 6.5, more preferably at a pH in the range of 5.5 to 6.0.

The copolymer (C) in the second coating comprises 25 to 60 mol-% methacrylic acid repeating units and 40 to 75 mol-% methyl methacrylate repeating units. The copolymer (C) preferably is an anionic copolymer. Preferably at least 90 mol-%, more preferably at least 95 mol-%, even more preferably at least 98 mol-%, of the repeating units in copolymer (C) are selected from methacrylic acid repeating units and methyl methacrylate repeating units. In particular, the copolymer (C) in the second coating may consist of methacrylic acid repeating units and methyl methacrylate repeating units. The copolymer (C) in the second coating preferably is a copolymer (C-1) or a copolymer (C-2), as described below. Accordingly, the second coating may comprise a copolymer (C-1), a copolymer (C-2), or the combination of both a copolymer (C-1) and a copolymer (C-2).

The copolymer (C-1) in the second coating comprises 25 to 60 mol-% methacrylic acid repeating units and 40 to 75 mol-% methyl methacrylate repeating units, preferably 45 to 55 mol-% methacrylic acid repeating units and 45 to 55 mol-% methyl methacrylate repeating units. Preferably at least 90 mol-%, more preferably at least 95 mol-%, even more preferably at least 98 mol-%, of the repeating units in copolymer (C-1) are selected from methacrylic acid repeating units and methyl methacrylate repeating units. For example, the copolymer (C-1) in the second coating may comprise methacrylic acid repeating units and methyl methacrylate repeating units in a molar ratio of 0.5:1 to 1.5:1, preferably in a molar ratio of 0.8:1 to 1:08, more preferably in a molar ratio of 1:1. In particular, the copolymer (C-1) in the second coating may consist of methacrylic acid repeating units and methyl methacrylate repeating units. A corresponding preferred example of copolymer (C-1) is poly(methacrylic acid-co-methyl methacrylate) 1:1, particularly Eudragit L 100 or Eudragit L 12.5.

The copolymer (C-2) in the second coating comprises 25 to 60 mol-% methacrylic acid repeating units and 40 to 75 mol-% methyl methacrylate repeating units, preferably 25 to 40 mol-% methacrylic acid repeating units and 60 to 75 mol-% methyl methacrylate repeating units. Accordingly, it is preferred that the copolymer (C-2) in the second coating comprises methacrylic acid repeating units and methyl methacrylate repeating units in a molar ratio of 1:1.5 to 1:2.5, more preferably in a molar ratio of 1:2. Preferably at least 90 mol-%, more preferably at least 95 mol-%, even more preferably at least 98 mol-%, of the repeating units in copolymer (C-2) are selected from methacrylic acid repeating units and methyl methacrylate repeating units. In particular, the copolymer (C-2) in the second coating may consist of methacrylic acid repeating units and methyl methacrylate repeating units. A corresponding preferred example of copolymer (C-2) is poly(methacrylic acid-co-methyl methacrylate) 1:2, particularly Eudragit S 100.

It is generally preferred that the copolymer (C) in the second coating, including the copolymer (C-1) and/or the copolymer (C-2), does not comprise methyl acrylate repeating units. Thus, the copolymer (C) in the second coating (or the copolymer (C-1) and/or the copolymer (C-2)) preferably comprises not more than 3 mol-% methyl acrylate repeating units, more preferably not more than 1 mol-%, even more preferably not more than 0.5 mol-%, yet even more preferably not more than 0.1 mol-%, still more preferably not more than 0.01 mol-%, most preferably 0 mol-% methyl acrylate repeating units.

It is preferred that the copolymer (C) in the second coating, including the copolymer (C-1) and/or the copolymer (C-2), is obtained from an aqueous dispersion of the respective copolymer (i.e., copolymer (C), copolymer (C-1) or copolymer (C-2)).

The second coating may further comprise one or more plasticizers. The one or more plasticizers are preferably selected from mono-, di- and tri-alkyl citrates such as, e.g., triethyl citrate, tripropyl citrate, tributyl citrate, or acetyl triethyl citrate; dialkyl sebacinates such as, e.g., diethyl sebacinate, dipropyl sebacinate, or dibutyl sebacinate; dialkyl phthalates such as, e.g., dimethyl phthalate, diethyl phthalate, dipropyl phthalate, dibutyl phthalate, or dioctyl phthalate; glycerol and mono-, di- and tri-glycerides such as, e.g., glyceryl triacetate, glyceryl tributyrate, glyceryl monostearate, or acetylated monoglycerides; propylene glycols and polyethylene glycols such as, e.g., PEG 300, PEG 400, PEG 600, PEG 800, PEG 1450, or PEG3350; fatty acids such as, e.g., stearic acid, oleic acid, or esters of fatty acids. More preferably, the one or more plasticizers are selected from mono-, di- and tri-alkyl citrates such as, e.g., triethyl citrate, tripropyl citrate, tributyl citrate, or acetyl triethyl citrate. Even more preferably, the second coating further comprises 10 to 80% by weight, preferably 40 to 80% by weight, of one or more selected from triethyl citrate, tripropyl citrate and tributyl citrate, based on the total weight of the second coating. An example of a preferred plasticizer is PlasACRYL, such as PlasACRYL™ HTP20 and PlasACRYL™ T20.

The second coating may, for example, account for at least 0.1% w/w, preferably 0.5 to 8% w/w, more preferably 1 to 5% w/w, in relation to the total weight of the solid oral pharmaceutical composition.

Optional Intermediate Coating

The solid oral pharmaceutical composition may further comprise one or more intermediate coatings located between the core and the first coating. The intermediate coating, or the innermost intermediate coating (in the case of more than one intermediate coating), may be a substantially continuous layer surrounding and containing the core of the solid oral pharmaceutical composition. Each intermediate coating (if present) preferably constitutes 5% w/w or less, more preferably 2% w/w or less, even more preferably 1% w/w or less of the solid oral pharmaceutical composition. Moreover, each intermediate coating (if present) preferably constitutes 0.1% w/w or more, more preferably 0.5% w/w or more of the solid oral pharmaceutical composition. The invention relates to all combinations of the aforementioned minimum and maximum contents by weight of the intermediate coating(s).

Each intermediate coating preferably comprises one or more polymers selected from ethyl cellulose, hydroxypropyl methylcellulose (HPMC), and polyvinyl acetate. More preferably, there is only one intermediate coating, and said intermediate coating comprises hydroxypropyl methylcellulose (HPMC). Even more preferably, there is only one intermediate coating, and said intermediate coating consists of hydroxypropyl methylcellulose (HPMC).

Optional Third Coating

The solid oral pharmaceutical composition may further comprise a third coating surrounding and containing the second coating (if present) or the first coating (if no second coating is present).

The third coating, if present, preferably accounts for at least 0.1% w/w, more preferably 0.5 to 8% w/w, even more preferably 1 to 5% w/w, of the total weight of the solid oral pharmaceutical composition.

The composition of the third coating is not particularly limited. Preferably, the third coating contains one or more copolymers selected from copolymers (A), (B), (C) and (D) as defined herein. More preferably, it comprises a cationic copolymer based on dimethylaminoethyl methacrylate, butyl methacrylate, and methyl methacrylate, preferably in a molar ratio of 2:1:1 (such as Eudragit E 100 or other Eudragit E polymers).

Alternatively, the third coating may be a top coat. The top coat may be a film coating or an immediate release coating. Examples of suitable top coats include Opadry® White (obtainable from Colorcon, Pa., USA), Opadry® II Yellow (obtainable from Colorcon, Pa., USA), or a copolymer based on methacrylic acid and ethyl acrylate, such as, e.g., a copolymer comprising at least 40% methacrylic acid repeating units and at least 40% ethyl acrylate repeating units. For example, the third coating may comprise poly(methacrylic acid-co-ethyl acrylate) 1:1.

The GLP-1 Receptor Agonist

The solid oral pharmaceutical composition of the present invention comprises a core which contains a GLP-1 receptor agonist. It is preferred that the GLP-1 receptor agonist is present only in the core, i.e. that it is not present in any coating comprised in the solid oral pharmaceutical composition.

The GLP-1 receptor agonist comprised in the solid oral pharmaceutical composition of the present invention is preferably a peptide. Accordingly, it is preferred that the GLP-1 receptor agonist is a peptidic GLP-1 receptor agonist.

More preferably, the GLP-1 receptor agonist is a peptide comprising or consisting of the sequence:

(SEQ ID NO: 1)
Xaa⁷-Xaa⁸-Glu-Gly-Thr-Xaa¹²-Thr-Ser-Asp-Xaa¹⁶-Ser-
Xaa¹⁸-Xaa¹⁹-Xaa²⁰-Glu-Xaa²²-Xaa²³-Xaa²⁴-Xaa²⁵-Xaa²⁶-
Lys-Phe-Ile-Xaa³⁰-Xaa³¹-Leu-Val-Xaa³⁴-Xaa³⁵-Xaa³⁶-
Xaa³⁷-Xaa³⁸-Xaa³⁹,

wherein:
Xaa⁷ is L-histidine, imidazopropionyl, α-hydroxy-histidine, D-histidine, deamino-histidine, 2-amino-histidine, β-hydroxy-histidine, homohistidine, Nα-acetyl-histidine, Nα-formyl-histidine, α-fluoromethyl-histidine, α-methyl-histidine, 3-pyridylalanine, 2-pyridylalanine, or 4-pyridylalanine;
Xaa⁸ is Ala, Gly, Val, Leu, Ile, Thr, Ser, Lys, α-aminoisobutyric acid, (1-aminocyclopropyl)carboxylic acid, (1-aminocyclobutyl)carboxylic acid, (1-aminocyclopentyl)carboxylic acid, (1-aminocyclohexyl)carboxylic acid, (1-aminocycloheptyl)carboxylic acid, or (1-aminocyclooctyl)carboxylic acid;
Xaa¹² is Lys or Phe;
Xaa¹⁶ is Val or Leu;
Xaa¹⁸ is Ser, Arg, Asn, Gln, or Glu;
Xaa¹⁹ is Tyr or Gln;
Xaa²⁶ is Leu, Lys, or Met;
Xaa²² is Gly, Glu, Lys, or α-aminoisobutyric acid;
Xaa²³ is Gln, Glu, or Arg;
Xaa²⁴ is Ala or Lys;
Xaa²⁵ is Ala or Val;
Xaa²⁶ is Val, His, Lys or Arg;
Xaa³⁶ is Ala, Glu, or Arg;
Xaa³¹ is Trp or His;
Xaa³⁴ is Glu, Asn, Gly, Gln, or Arg;
Xaa³⁵ is Gly, α-aminoisobutyric acid, or absent;
Xaa³⁶ is Arg, Gly, Lys, or absent;
Xaa³⁷ is Gly, Ala, Glu, Pro, Lys, Arg, or absent;
Xaa³⁸ is Ser, Gly, Ala, Glu, Gln, Pro, Arg, or absent; and
Xaa³⁹ is Gly or absent.

Even more preferably, the GLP-1 receptor agonist is selected from semaglutide, liraglutide, exenatide, albiglutide, dulaglutide, lixisenatide, taspoglutide, langlenatide, beinaglutide, efpeglenatide, GLP-1(7-37), GLP-1(7-36)NH₂, and oxyntomodulin. Still more preferably, the GLP-1 receptor agonist is selected from semaglutide, liraglutide, exenatide, albiglutide, dulaglutide, and lixisenatide.

The GLP-1 receptor agonist may also be a dual GLP-1 receptor/glucagon receptor agonist, a dual GLP-1 receptor/gastric inhibitory peptide (GIP) receptor agonist, or a triple GLP-1 receptor/GIP receptor/glucagon receptor agonist. The dual GLP-1 receptor/glucagon receptor agonist may be, e.g., any one of the compounds referred to in WO 2015/185640, WO 2015/086733 or WO 2015/155139, which are incorporated herein by reference. The dual GLP-1 receptor/GIP receptor agonist may be, e.g., any one of the compounds referred to in WO 2013/164483 or WO 2015/086728, which are incorporated herein by reference. In addition, the dual GLP-1 receptor/glucagon receptor agonist, the dual GLP-1 receptor/GIP receptor agonist or the triple GLP-1 receptor/GIP receptor/glucagon receptor agonist may each be a conjugate (particularly a protein or peptide) comprising any of the GLP-1 receptor agonists described herein covalently attached, optionally via a linker, to a glucagon receptor agonist and/or to a GIP receptor agonist.

Moreover, the GLP-1 receptor agonist may also be any one of the GLP-1 agonists disclosed in WO 93/19175, WO 96/29342, WO 98/08871, WO 99/43707, WO 99/43706, WO 99/43341, WO 99/43708, WO 2005/027978, WO 2005/058954, WO 2005/058958, WO 2006/005667, WO 2006/037810, WO 2006/037811, WO 2006/097537, WO 2006/097538, WO 2008/023050, WO 2009/030738, WO 2009/030771, or WO 2009/030774, each of which is incorporated herein by reference. The GLP-1 receptor agonist may furthermore be oxyntomodulin or a derivative or analog thereof.

Further preferred examples of the GLP-1 receptor agonist include, in particular, tirzepatide, a tirzepatide-like peptide (such as, e.g., LY3537031 or LY3493269), cotadutide, or BI 456906 (which is a GLP-1/glucagon dual agonist). A further exemplary GLP-1 receptor agonist is LY3502970 (GLP-1R NPA).

The GLP-1 receptor agonist comprised in the solid oral pharmaceutical composition of the present invention may further be used in combination with one or more other therapeutic agents, such as insulin (preferably human insulin) or an insulin analog (e.g., a long acting basal insulin analog or a protease stabilized long acting basal insulin analog; exemplary insulin analogs include, without limitation, insulin lispro, insulin PEGlispro, the insulin derivative “A14E, B25H, B29K(N(eps)octadecanedioyl-gGlu-OEG-OEG), desB30 human insulin” (see, e.g., US 2014/0056953 A1), insulin aspart, insulin glulisine, insulin glargine, insulin detemir, NPH insulin, insulin degludec, or the insulin analogs/derivatives described in US 2014/0056953 A1, which is incorporated herein by reference, particularly each one of the insulin analogs/derivatives described in paragraphs [0225] to [0332] of US 2014/0056953 A1). The insulin or the insulin analog may be present in the solid oral pharmaceutical composition according to the invention, preferably in the core of the solid oral pharmaceutical composition, or may be provided in a separate pharmaceutical composition. In the latter case, the separate pharmaceutical composition comprising insulin or an insulin analog may be administered simultaneously/concomitantly with the solid oral pharmaceutical composition (comprising the GLP-1 receptor agonist), or may be administered sequentially. It is preferred that the insulin or the insulin analog is provided in the core of the solid oral pharmaceutical composition according to the invention (together with the GLP-1 receptor agonist).

It will be understood that the GLP-1 receptor agonist may be present in non-salt form or in the form of a pharmaceutically acceptable salt. A corresponding pharmaceutically acceptable salt may be formed, e.g., by protonation of an atom carrying an electron lone pair which is susceptible to protonation, such as an amino group, with an inorganic or organic acid, or as a salt of a carboxylic acid group with a physiologically acceptable cation as they are well-known in the art. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts, nitrate salts, phosphate salts (such as, e.g., phosphate, hydrogenphosphate, or dihydrogenphosphate salts), carbonate salts, hydrogencarbonate salts or perchlorate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, glycolate, nicotinate, benzoate, salicylate, ascorbate, or pamoate (embonate) salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; and acidic amino acid salts such as aspartate or glutamate salts.

The solid oral pharmaceutical composition may optionally comprise one or more further pharmaceutically acceptable excipients, such as carriers, diluents, fillers, disintegrants, lubricating agents, binders, colorants, pigments, stabilizers, preservatives, antioxidants, amino acids, reducing agents, bioadhesive agents and/or solubility enhancers. In particular, it may comprise one or more additives selected from vitamin E, histidine, microcrystalline cellulose (MCC), mannitol, starch, sorbitol and/or lactose. The solid oral pharmaceutical composition can be formulated by techniques known to the person skilled in the art, such as the techniques published in Remington's Pharmaceutical Sciences, 20th Edition.

As noted above, the solid oral pharmaceutical composition may comprise one or more solubility enhancers, such as, e.g., poly(ethylene glycol), including poly(ethylene glycol) having a molecular weight in the range of about 200 to about 5,000 Da, ethylene glycol, propylene glycol, non-ionic surfactants, tyloxapol, polysorbate 20, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate, or any combination thereof.

Moreover, as noted above, the solid oral pharmaceutical composition may comprise one or more pharmaceutically acceptable carriers. The pharmaceutically acceptable carrier may be an aqueous or non-aqueous agent, for example alcoholic or oleaginous, or a mixture thereof, and may contain a surfactant, an emollient, a lubricant, a stabilizer, a dye, a perfume, a preservative, an acid or base for adjustment of pH, a solvent, an emulsifier, a gelling agent, a moisturizer, a stabilizer, a wetting agent, a time release agent, a humectant, or any other component commonly included in a particular form of solid oral pharmaceutical composition. Pharmaceutically acceptable carriers are well known in the art and include, for example, aqueous solutions such as water or physiologically buffered saline or other solvents or vehicles such as glycols, glycerol, and oils such as olive oil or injectable organic esters. A pharmaceutically acceptable carrier can contain physiologically acceptable compounds that act, for example, to stabilize or to increase the absorption of the GLP-1 receptor agonist, for example, carbohydrates, such as glucose, sucrose or dextrans, antioxidants, such as ascorbic acid or glutathione, chelating agents, low molecular weight proteins or other stabilizers or excipients. A pharmaceutically acceptable carrier can also be selected from substances such as distilled water, benzyl alcohol, lactose, starches, talc, magnesium stearate, polyvinylpyrrolidone, alginic acid, colloidal silica, titanium dioxide, and flavoring agents. Preferred pharmaceutically acceptable carriers, in particular for use in the core of the solid oral pharmaceutical composition of the present invention, are selected from microcrystalline cellulose, mannitol, starch, sorbitol and lactose.

The Core of the Solid Oral Pharmaceutical Composition

The core of the solid oral pharmaceutical composition of the present invention comprises a GLP-1 receptor agonist (as described above).

It is preferred that the core of the solid oral pharmaceutical composition further comprises a permeation enhancer (also referred to as a “mucosal permeation enhancer”). The use of a permeation enhancer improves or facilitates the mucosal absorption/permeation of the GLP-1 receptor agonist, particularly through the intestinal mucosa.

The permeation enhancer may be, e.g., a zwitter-ionic permeation enhancer, a cationic permeation enhancer, an anionic permeation enhancer (e.g., an anionic permeation enhancer comprising one or more sulfonic acid groups (—SO₃H)), or a non-ionic permeation enhancer.

It is preferred that the permeation enhancer is selected from C₈₋₂₀ alkanoyl carnitine (preferably lauroyl carnitine, myristoyl carnitine or palmitoyl carnitine; e.g., lauroyl carnitine chloride, myristoyl carnitine chloride or palmitoyl carnitine chloride), salicylic acid (preferably a salicylate, e.g., sodium salicylate), a salicylic acid derivative (such as, e.g., 3-methoxysalicylic acid, 5-methoxysalicylic acid, or homovanillic acid, a C₈₋₂₀ alkanoic acid (preferably a C₈₋₂₀ alkanoate, more preferably a caprate, a caprylate, a myristate, a palmitate, or a stearate, such as, e.g., sodium caprate, sodium caprylate, sodium myristate, sodium palmitate, or sodium stearate), citric acid (preferably a citrate, e.g., sodium citrate), tartaric acid (preferably a tartrate), a fatty acid acylated amino acid (e.g., any of the fatty acid acylated amino acids described in US 2014/0056953 A1 which is incorporated herein by reference, including, without being limited thereto, sodium lauroyl alaninate, N-dodecanoyl-L-alanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamic acid, N-dodecanoyl-L-glutamic acid, sodium lauroyl glutaminate, N-dodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyl-L-isoleucine, sodium lauroyl leucinate, N-dodecanoyl-L-leucine, sodium lauroyl methioninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodium lauroyl tryptophanate, N-dodecanoyl-L-tryptophane, sodium lauroyl tyrosinate, N-dodecanoyl-L-tyrosine, sodium lauroyl valinate, N-dodecanoyl-L-valine, sodium lauroyl sarcosinate, N-dodecanoyl-L-sarcosine, sodium capric alaninate, N-decanoyl-L-alanine, sodium capric asparaginate, N-decanoyl-L-asparagine, sodium capric aspartic acid, N-decanoyl-L-aspartic acid, sodium capric cysteinate, N-decanoyl-L-cysteine, sodium capric glutamic acid, N-decanoyl-L-glutamic acid, sodium capric glutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-L-leucine, sodium capric methioninate, N-decanoyl-L-methionine, sodium capric phenylalaninate, N-decanoyl-L-phenylalanine, sodium capric prolinate, N-decanoyl-L-proline, sodium capric serinate, N-decanoyl-L-serine, sodium capric threoninate, N-decanoyl-L-threonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, N-decanoyl-L-sarcosine, sodium oleoyl sarcosinate, sodium N-decylleucine, sodium stearoyl glutamate (e.g., Amisoft HS-11 P), sodium myristoyl glutamate (e.g., Amisoft MS-11), sodium lauroyl glutamate (e.g., Amisoft LS-11), sodium cocoyl glutamate (e.g., Amisoft CS-11), sodium cocoyl glycinate (e.g., Amilite GCS-11), sodium N-decyl leucine, sodium cocoyl glycine, sodium cocoyl glutamate, sodium lauroyl alaninate, N-dodecanoyl-L-alanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamic acid, N-dodecanoyl-L-glutamic acid, sodium lauroyl glutaminate, N-dodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyl-L-isoleucine, sodium lauroyl leucinate, N-dodecanoyl-L-leucine, sodium lauroyl methinoninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodium lauroyl tryptophanate, N-dodecanoyl-L-tryptophane, sodium lauroyl tyrosinate, N-dodecanoyl-L-tyrosine, sodium lauroyl valinate, N-dodecanoyl-L-valine, N-dodecanoyl-L-sarcosine, sodium capric alaninate, N-decanoyl-L-alanine, sodium capric asparaginate, N-decanoyl-L-asparagine, sodium capric aspartic acid, N-decanoyl-L-aspartic acid, Sodium capric cysteinate, N-decanoyl-L-cysteine, sodium capric glutamic acid, N-decanoyl-L-glutamic acid, sodium capric glutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-L-leucine, leucine, sodium capric methioninate, N-decanoyl-L-methionine, sodium capric phenylalaninate, N-decanoyl-L-phenylalanine, sodium capric prolinate, N-decanoyl-L-proline, sodium capric serinate, N-decanoyl-L-serine, sodium capric threoninate, N-decanoyl-L-threonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, sodium oleoyl sarcosinate, and pharmaceutically acceptable salts of any of the aforementioned compounds; or, e.g., C₈₋₂₀ alkanoyl sarcosinate (e.g., a lauroyl sarcosinate, such as sodium lauroyl sarcosinate) or one of the 20 standard proteinogenic α-amino acids that is acylated with a C₈₋₂₀ alkanoic acid), an alkylsaccharide (e.g., a C₁₋₂₀ alkylsaccharide, such as, e.g., C₈₋₁₀ alkylpolysaccharide like Multitrope™ 1620-LQ-(MV); or, e.g., n-octyl-beta-D-glucopyranoside, n-dodecyl-beta-D-maltoside, n-tetradecyl-beta-D-maltoside, tridecyl-beta-D-maltoside, sucrose laurate, sucrose stearate, sucrose myristate, sucrose palmitate, sucrose cocoate, sucrose mono-dodecanoate, sucrose mono-tridecanoate, sucrose mono-tetradecanoate, a coco-glucoside, or any of the alkylsaccharides described in U.S. Pat. No. 5,661,130 or in WO 2012/112319 which are herein incorporated by reference), a cyclodextrine (e.g., α-cyclodextrin, β-cyclodextrin, γ-cyclodextrin, methyl-β-cyclodextrin, hydroxypropyl β-cyclodextrin, or sulfobutylether cyclodextrin), N-[8-(2-hydroxybenzoyl)amino]caprylic acid (preferably a N-[8-(2-hydroxybenzoyl)amino]caprylate, more preferably sodium N-[8-(2-hydroxybenzoyl)amino]caprylate, also referred to as “SNAG”), a N-[8-(2-hydroxybenzoyl)amino]caprylate derivative (preferably a sodium N-[8-(2-hydroxybenzoyl)amino]caprylate derivative), a thiomer (also referred to as a thiolated polymer; may be synthesized, e.g., by immobilization of sulfhydryl bearing ligands on a polymeric backbone of well-established polymers such as, e.g., polyacrylic acid, carboxymethylcellulose or chitosan; exemplary thiomers include the thiomers that are described in Laffleur F et al., Future Med Chem. 2012, 4(17):2205-16 (doi: 10.4155/fmc.12.165) which is incorporated herein by reference), a mucoadhesive polymer having a vitamin B partial structure (e.g., any of the mucoadhesive polymers described in U.S. Pat. No. 8,980,238 B2 which is incorporated herein by reference; including, in particular, any of the polymeric compounds as defined in any one of claims 1 to 3 of U.S. Pat. No. 8,980,238 B2), a calcium chelating compound (e.g., ethylenediaminetetraacetic acid (EDTA), ethylene glycol tetraacetic acid (EGTA), sodium citrate, or polyacrylic acid), cremophor EL (also referred to as “Kolliphor EL”; CAS no. 61791-12-6), chitosan, N,N,N-trimethyl chitosan, benzalkonium chloride, bestatin, cetylpyridinium chloride, cetyltrimethylammonium bromide, a C₂₋₂₀ alkanol (e.g., ethanol, decanol, lauryl alcohol, myristyl alcohol, or palmityl alcohol), a C₈₋₂₀ alkenol (e.g., oleyl alcohol), a C₈₋₂₀ alkenoic acid (e.g., oleic acid), dextran sulfate, diethyleneglycol monoethyl ether (transcutol), 1-dodecylazacyclo-heptan-2-one (Atone), caprylocaproyl polyoxylglycerides (such as, e.g., caprylocaproyl polyoxyl-8 glycerides; available, e.g., as Labrasol® or ACCONON® MC8-2), ethyl caprylate, glyceryl monolaurate, lysophosphatidylcholine, menthol, a C₈₋₂₀ alkylamine, a C₈₋₂₀ alkenylamine (e.g., oleylamine), phosphatidylcholine, a poloxamer, polyethylene glycol monolaurate, polyoxyethylene, polypropylene glycol monolaurate, a polysorbate (e.g., polysorbate 20 or polysorbate 80), cholic acid (preferably a cholate, e.g., sodium chlolate), a deoxycholate (e.g., sodium deoxycholate), a chenodeoxycholate (e.g., sodium chenodeoxycholate), sodium glycocholate, sodium glycodeoxycholate, sodium lauryl sulfate (SDS), sodium decyl sulfate, sodium octyl sulfate, sodium laureth sulfate, N-lauryl sarcosinate, decyltrimethyl ammonium bromide, benzyldimethyl dodecyl ammonium chloride, myristyltrimethyl ammonium chloride, dodecyl pyridinium chloride, decyldimethyl ammonio propane sulfonate, myristyldimethyl ammonio propane sulfonate, palmityldimethyl ammonio propane sulfonate, ChemBetaine CAS, ChemBetaine Oleyl, Nonylphenoxypolyoxyethylene, polyoxyethylene sorbitan monolaurate, polyoxyethylene sorbitan monopalmitate, sorbitan monooleate, Triton X-100, hexanoic acid, heptanoic acid, methyl laurate, isopropyl myristate, isopropyl palmitate, methyl palmitate, diethyl sebaccate, sodium oleate, urea, lauryl amine, caprolactam, methyl pyrrolidone, octyl pyrrolidone, methyl piperazine, phenyl piperazine, Carbopol 934P, glyccyrhetinic acid, bromelain, pinene oxide, limonene, cineole, octyl dodecanol, fenchone, menthone, trimethoxy propylene methyl benzene, a cell-penetrating peptide (e.g., Klaklak, polyarginine or oligoarginine (particularly octa-arginine), penetratin (particularly L-penetratin), a penetratin analog (particularly PenetraMax; see, e.g., El-Sayed Khafagy et al., Eur J Pharm Biopharm. 2013; 85(3 Pt A):736-43), HIV-1 Tat, transportan, or any of the cell-penetrating peptides referred to in US 2012/0065124), macrogol-15-hydroxystearate (e.g., Solutol HS 15), CriticalSorb (see., e.g., Ilium L et al. J Control Release. 2012; 162(1):194-200), a taurocholate (e.g., sodium taurocholate), a taurodeoxycholate (e.g., sodium taurodeoxycholate), a sulfoxide (e.g., a (C₁₋₁₀ alkyl)-sulfoxide, such as, e.g., decyl methyl sulfoxide, or dimethyl sulfoxide), cyclopentadecalactone, 8-(N-2-hydroxy-5-chloro-benzoyl)-amino-caprylic acid (also referred to as “5-CNAC”), N-(10-[2-hydroxybenzoyl]amino)decanoic acid (also referred to as “SNAD”), dodecyl-2-N,N-dimethylamino propionate (also referred to as “DDAIP”), D-α-tocopheryl polyethylene glycol-1000 succinate (also referred to as “TPGS”), arginine, and pharmaceutically acceptable salts of the aforementioned compounds. Mixtures of two or more permeation enhancers, including any of the above-described permeation enhancers, can also be used. Moreover, any of the chemical permeation enhancers described in Whitehead K et al. Pharm Res. 2008 June; 25(6):1412-9 (particularly any one of those described in Table I of this reference), any one of the modified amino acids disclosed in U.S. Pat. No. 5,866,536 (particularly any one of compounds I to CXXIII, as disclosed in U.S. Pat. No. 5,866,536 which is incorporated herein by reference, or a pharmaceutically acceptable salt or solvate thereof, such as a disodium salt, an ethanol solvate, or a hydrate of any one of these compounds), any one of the modified amino acids disclosed in U.S. Pat. No. 5,773,647 (particularly any one of compounds 1 to 193, as disclosed in U.S. Pat. No. 5,773,647 which is incorporated herein by reference, or a pharmaceutically acceptable salt or solvate thereof, such as a disodium salt, an ethanol solvate, or a hydrate of any one of these compounds), any of the nanoparticles described in WO 2011/133198, any of the polymer preparations described in US 2015/174076 and/or a hydrogel (e.g., as described in Torres-Lugo M et al. Biotechnol Prog. 2002; 18(3):612-6) can likewise be used as permeation enhancer. Moreover, a complex lipoidal dispersion (e.g., a combination of an insoluble surfactant or oil with a soluble surfactant, and optionally with water or a co-solvent) can also be used as permeation enhancer; corresponding exemplary permeation enhancers include, in particular, mixed micelles, reversed micelles, a self emulsifying system (e.g., SEDDS, SMEDDS, or SNEDDS), a lipid dispersion, a course emulsion, or solid lipid nanoparticles (SLNs). Preferably, the permeation enhancer is selected from sodium caprylate, sodium caprate, sodium laurate, sucrose laurate, sucrose stearate, sodium stearate, EDTA, polyacrylic acid, and N-[8-(2-hydroxybenzoyl)amino]caprylate or a pharmaceutically acceptable salt thereof (particularly sodium N-[8-(2-hydroxybenzoyl)amino]caprylate). A particularly preferred permeation enhancer is N-[8-(2-hydroxybenzoyl)amino]caprylate or a pharmaceutically acceptable salt thereof, in particular sodium N-[8-(2-hydroxybenzoyl)amino]caprylate.

Further preferred permeation enhancers are alkyl polysaccharides, arginine or CriticalSorb® (Solutol® HS15). In particular, the permeation enhancer may an alkyl glycoside (or a combination of two or more alkyl glycosides) which may be selected from any of the alkyl glycosides described in the following.

Alkyl glycosides to be used as permeation enhancer in accordance with the present invention can be synthesized by known procedures, i.e., chemically, as described, e.g., in Rosevear et al., Biochemistry 19:4108-4115 (1980) or Koeltzow and Urfer, J. Am. Oil Chem. Soc., 61:1651-1655 (1984), U.S. Pat. No. 3,219,656 or U.S. Pat. No. 3,839,318 or enzymatically, as described, e.g., in Li et al., J. Biol. Chem., 266:10723-10726 (1991) or Gopalan et al., J. Biol. Chem. 267:9629-9638 (1992).

Alkyl glycosides to be used as permeation enhancer in the present invention can include, but are not limited to: alkyl glycosides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside (which may be synthesized according to Koeltzow and Urfer; Anatrace Inc., Maumee, Ohio; Calbiochem, San Diego, Calif.; Fluka Chemie, Switzerland); alkyl thiomaltosides, such as heptyl-, octyl-, dodecyl-, tridecyl-, and tetradecyl-β-D-thiomaltoside (which may be synthesized according to Defaye, J. and Pederson, C., “Hydrogen Fluoride, Solvent and Reagent for Carbohydrate Conversion Technology” in Carbohydrates as Organic Raw Materials, 247-265 (F. W. Lichtenthaler, ed.) VCH Publishers, New York (1991); Ferenci, T., J. Bacteriol, 144:7-11 (1980)); alkyl thioglucosides, such as heptyl- or octyl 1-thio α- or β-D-glucopyranoside (Anatrace, Inc., Maumee, Ohio; see Saito, S. and Tsuchiya, T. Chem. Pharm. Bull. 33:503-508 (1985)); alkyl thiosucroses (which may be synthesized according to, for example, Binder, T. P. and Robyt, J. F., Carbohydr. Res. 140:9-20 (1985)); alkyl maltotriosides (which may be synthesized according to Koeltzow and Urfer); long chain aliphatic carbonic acid amides of sucrose β-amino-alkyl ethers (which may be synthesized according to Austrian Patent 382,381 (1987); Chem. Abstr., 108:114719 (1988) and Gruber and Greber pp. 95-116); derivatives of palatinose and isomaltamine linked by amide linkage to an alkyl chain (which may be synthesized according to Kunz, M., “Sucrose-based Hydrophilic Building Blocks as Intermediates for the Synthesis of Surfactants and Polymers” in Carbohydrates as Organic Raw Materials, 127-153); derivatives of isomaltamine linked by urea to an alkyl chain (which may be synthesized according to Kunz); long chain aliphatic carbonic acid ureides of sucrose β-amino-alkyl ethers (which may be synthesized according to Gruber and Greber, pp. 95-116); and long chain aliphatic carbonic acid amides of sucrose β-amino-alkyl ethers (which may be synthesized according to Austrian Patent 382,381 (1987), Chem. Abstr., 108:114719 (1988) and Gruber and Greber, pp. 95-116).

The permeation enhancer may also be selected from any of the enhancing agents referred to in U.S. Pat. No. 8,927,497, including in particular any of alkyl glycosides, any of the saccharide alkyl esters, and/or any of the mucosal delivery-enhancing agents described in this document.

Moreover, the permeation enhancer may also be a compound of the following formula (I):

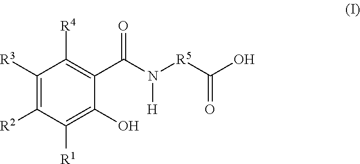

wherein:
R¹, R², R³ and R⁴ are each independently selected from hydrogen, —OH, —NR₆R⁷, halogen (e.g., —F, —Cl, —Br or —I), C₁₋₄ alkyl or C₁₋₄ alkoxy;
R⁵ is a substituted or unsubstituted C₂₋₁₆ alkylene, substituted or unsubstituted C₂₋₁₆ alkenylene, substituted or unsubstituted C₁₋₁₂ alkyl(arylene) [e.g., substituted or unsubstituted C₁₋₁₂ alkyl(phenylene)], or substituted or unsubstituted aryl(C₁₋₁₂ alkylene) [e.g., substituted or unsubstituted phenyl(C₁₋₁₂ alkylene)]; and
R⁶ and R⁷ are each independently hydrogen, oxygen, —OH or C₁₋₄ alkyl;
or a pharmaceutically acceptable salt or solvate thereof, particularly a disodium salt, an alcohol solvate (e.g., a methanol solvate, an ethanol solvate, a propanol solvate, or a propylene glycol solvate, or any such solvate of the disodium salt; particularly an ethanol solvate or an ethanol solvate of the disodium salt), or a hydrate thereof (e.g., a monohydrate of the disodium salt). The above-mentioned “substituted” groups comprised in formula (I) are preferably substituted with one or more (e.g., one, two, or three) substituent groups independently selected from halogen (e.g., —F, —Cl, —Br or —I), —OH, C₁₋₄ alkyl or C₁₋₄ alkoxy. Such compounds and methods for their preparation are described, e.g., in WO 00/59863 which is incorporated herein by reference. Accordingly, the permeation enhancer may also be a “delivery agent” as described in WO 00/59863. Preferred examples of the compounds of formula (I) include N-(5-chlorosalicyloyl)-8-aminocaprylic acid, N-(10-[2-hydroxybenzoyliamino)decanoic acid, N-(8-[2-hydroxybenzoyl]amino)caprylic acid, a monosodium or disodium salt of any one of the aforementioned compounds, an ethanol solvate of the sodium salt (e.g., monosodium or disodium salt) of any one of the aforementioned compounds, a monohydrate of the sodium salt (e.g., monosodium or disodium salt) of any one of the aforementioned compounds, and any combination thereof. A particularly preferred compound of formula (I) is the disodium salt of N-(5-chlorosalicyloyl)-8-aminocaprylic acid or the monohydrate thereof.

It is particularly preferred that the permeation enhancer is selected from sodium caprate, sodium caprylate, mixtures of sodium caprate and sodium caprylate, SNAC, sucrose laurate, labrasol and polysorbate.

Furthermore, the permeation enhancer may also be a salt of a medium-chain fatty acid. The salt of a medium-chain fatty acid is preferably a salt of a C₄₋₁₈ saturated fatty acid, preferably a C₄₋₁₈ linear or branched alkanoic acid which optionally has 1, 2 or 3 C═C double bonds, more preferably a C₆₋₁₆ linear or branched alkanoic acid which optionally has 1, 2 or 3 C═C double bonds, even more preferably a C₆₋₁₄ linear or branched alkanoic acid which optionally has 1, 2 or 3 C═C double bonds. The salt of a medium-chain fatty acid is preferably a salt of a 04-18 linear or branched alkanoic acid, more preferably a C₆₋₁₆ linear or branched alkanoic acid, even more preferably a C₆₋₁₄ linear or branched alkanoic acid. The salt of a medium-chain fatty acid is preferably selected from a salt of a valeric acid, caproic acid, enanthic acid, caprylic acid, pelargonic acid, capric acid, undecylic acid, lauric acid, tridecylic acid, myristic acid, pentadecylic acid, palmitic acid, margaric acid and/or stearic acid. More preferably, the salt of a medium-chain fatty acid is a salt of capric acid.

The salt of a medium-chain fatty acid is more preferably a sodium salt or a potassium salt. It is furthermore preferred that the salt of a medium-chain fatty acid is a salt of capric acid. Capric acid may also be referred to as decanoic acid (CH₃(CH₂)₈COOH). A preferred salt of capric acid is sodium caprate (i.e. CH₃(CH₂)₈COONa).

The solid oral pharmaceutical composition of the present invention preferably further comprises one or more enzyme inhibitors. Preferred enzyme inhibitors include trisodium phosphate (Na₃PO₄), arginine and lysine.

More preferably, the solid oral pharmaceutical composition of the present invention comprises a combination of sodium caprate and trisodium phosphate. Still more preferably, the core of the solid oral pharmaceutical composition of the present invention comprises sodium caprate, trisodium phosphate and the GLP-1 receptor agonist.

Method of Preparing the Solid Oral Pharmaceutical Composition

The present invention furthermore relates to a method for preparing the solid oral pharmaceutical composition of the present invention. The method preferably comprises the steps of
preparing a core, and
applying the first coating completely surrounding the core.

Preferably, the first coating is applied using a first aqueous composition comprising a copolymer (A) in combination with a copolymer (B) and/or a copolymer (C) and/or a copolymer (D). More preferably, the first coating is applied using a first aqueous composition comprising a copolymer (A) in combination with a copolymer (B) and/or a copolymer (C).

The first aqueous composition preferably further comprises an anti-tacking agent. The anti-tacking agent is preferably selected from glycerol monostearate, talc or PlasAcryl.

In addition, the first aqueous composition preferably further comprises citric acid and/or has a pH in a range of 2 to 5, preferably 3 to 4.

Preferably, the method further comprises a step of applying the second coating completely surrounding the first coating, wherein the second coating is applied using a second aqueous composition comprising the copolymer (C) as defined above. The second aqueous composition optionally further comprises an anti-tacking agent, wherein the anti-tacking agent is preferably selected from glycerol monostearate and talc.

The method may further include applying any one or more of the other coatings described herein above, preferably by using an aqueous dispersion comprising the polymers and/copolymers and other intended components of the respective coating(s). It has been found that copolymers such as poly(methacrylic acid-co-methyl methacrylate) 1:2 (such as Eudragit S 100) may already dissolve at a pH of less than 7, in particular at a pH of about 6.5, when applied from an aqueous dispersion.

Dosage Forms

The solid oral pharmaceutical composition of the present invention is preferably an oral dosage form, more preferably a peroral dosage form.

Accordingly, the solid oral pharmaceutical composition is preferably to be administered orally, particularly perorally (or is formulated for oral administration, particularly peroral administration). Even more preferably, the solid oral pharmaceutical composition is to be administered by oral ingestion, particularly by swallowing. The solid oral pharmaceutical composition can thus be administered to pass through the mouth into the gastrointestinal tract, which can also be referred to as “oral-gastrointestinal” administration.

In a preferred embodiment, the solid oral pharmaceutical composition is to be administered perorally (particularly by oral-gastrointestinal administration) to a subject/patient in fed state.

Accordingly, it is preferred that the solid oral pharmaceutical composition is administered perorally after a meal, i.e. after the intake of food (e.g., within about 1 to 2 hours after a meal). In a further preferred embodiment, the solid oral pharmaceutical composition is to be administered perorally (particularly by oral-gastrointestinal administration) to a subject/patient after overnight fasting together with a meal. Thus, it is preferred that the solid oral pharmaceutical composition is administered perorally together with food (e.g., breakfast) in the morning, particularly after overnight fasting.

In particular, it is preferred that the solid oral pharmaceutical composition is in the form of a capsule or a tablet. The total weight of the solid oral pharmaceutical composition, such as the capsule or tablet, may be in the range of 100 mg to 1500 mg. The total weight of the solid oral pharmaceutical composition is more preferably in the range of 100 mg to 1200 mg, 200 mg to 1000 mg, 400 mg to 800 mg, or 600 mg to 900 mg. A tablet or capsule preferably has a total weight of at least 100 mg, such as 100 mg to 1200 mg, 400 mg to 800 mg or 600 mg to 900 mg.

Moreover, the core may be in the form of a multiparticulate. The term “multiparticulate” preferably refers to particles having a volume mean particle size, as determined by laser diffraction, of 0.05 to 2 mm. For example, the GLP-1 receptor agonist may be present in the form of a multitude of particles within a matrix of the permeation enhancer. More preferably, the core is in the form of mini-tablets, a granulate or pellets. The core may also be a so-called robotic capsule (e.g., a RaniPill™) comprising the GLP-1 receptor agonist in form of microneedles and an injection device inside the capsule.

Even more preferably, the solid oral pharmaceutical composition is formulated as a peroral dosage form for release of the GLP-1 receptor agonist in the small intestine and/or colon. Release in the ileum is more preferred. Coatings as set out in the present invention allow for a delayed release of the GLP-1 receptor agonist primarily in the lower part of the small intestine. In contrast to conventional solid oral pharmaceutical compositions based (mainly) on anionic polymers which dissolve at a pH of more than 7, the delayed release by the solid oral pharmaceutical compositions of the present invention is regulated by non-ionic pH-independent polymers. As a result, the small intestinal transit time is more constant and lower variations in drug release are observed, as compared to coatings mainly containing pH-dependent polymers. In addition, dose dumping, which can occur if a subject does not reach or reaches only for a short time a pH of more than 7, can be avoided by the solid oral pharmaceutical compositions of the present invention.

It is preferred that the solid oral pharmaceutical composition according to the invention has a dissolution profile (dissolution method according to the United States Pharmacopeia, USP) with less than 5% of the GLP-1 receptor agonist released within 2 hours at acidic stage (simulated gastric fluid according to USP or 0.1 M HCl), followed by dissolution in simulated intestinal fluid at pH between 6 and 6.5 with a lag time of at least 1 hour (more preferably at least 1.5 hours, even more preferably at least 2 hours, yet even more preferably at least 2.5 hours). Within the lag time, not more than 10% of the GLP-1 receptor agonist is released. After the lag time, more than 75% of the GLP-1 receptor agonist is released in simulated intestinal fluid at pH between 6 and 6.5 within 1 hour. Accordingly, it is preferred that the solid oral pharmaceutical composition has a dissolution profile, as determined by the dissolution method according to USP, with less than 5% of the GLP-1 receptor agonist released within 2 hours in simulated gastric fluid, followed by dissolution in simulated intestinal fluid at pH between 6 and 6.5 with a lag time of at least 1 hour, whereby not more than 10% of the GLP-1 receptor agonist is released within the lag time, and whereby after the lag time more than 75% of the GLP-1 receptor agonist is released in simulated intestinal fluid at pH between 6 and 6.5 within 1 hour. As explained above, the dissolution profile should be determined in accordance with the US Pharmacopeia (USP, preferably in the version as of Sep. 1, 2020); alternatively, however, the dissolution profile may also be determined using the modified version of the USP dissolution method described herein below in Example 7. Exemplary solid oral pharmaceutical compositions according to the present invention having such a dissolution profile are described below in the examples section.

Further examples of solid oral pharmaceutical compositions according to the invention, having the above-described preferred dissolution profile, include compositions with any of the following features:
first coating comprising HPMC (such as, e.g., Methocel LV), followed by a second coating (which is exterior to the first coating) which is a pH dependent enteric coating (such as, e.g., Eudragit L30 D-55, Eudragit L100, or Eudragit S100), wherein the first coating preferably has a coating thickness between 200 μm and 700 μm (more preferably between 300 and 600 μm); or
first coating comprising a combination of ethylcellulose and sodium alginate (such as, e.g., Nutrateric® from Colorcon), followed by a second coating (which is exterior to the first coating) which is a pH dependent enteric coating (such as, e.g., Eudragit L30 D-55, Eudragit L100, or Eudragit S100); or
first coating comprising polyvinyl alcohol (PVA) or a PVA based film coating, followed by a second coating (which is exterior to the first coating) which is a pH dependent enteric coating (such as, e.g., Eudragit L30 D-55, Eudragit L100, or Eudragit S100); or
first coating comprising a polyvinyl alcohol-polyethylene glycol (PVA-PEG) graft copolymer or a PVA-PEG graft copolymer based film coating (such as, e.g., Opadry QX from Colorcon), followed by a second coating (which is exterior to the first coating) which is a pH dependent enteric coating (such as, e.g., Eudragit L30 D-55, Eudragit L100, or Eudragit S100).

Furthermore, it is preferred that the solid oral pharmaceutical composition according to the invention has a dissolution profile (dissolution method according to the United States Pharmacopeia, USP) with less than 5% of the GLP-1 receptor agonist released within 1 hour at acidic stage (simulated gastric fluid according to USP or 0.1 M HCl), followed by dissolution in simulated intestinal fluid at pH between 6 and 6.5 with a lag time of at least 1 hour (more preferably at least 1.5 hours, even more preferably at least 2 hours, yet even more preferably at least 2.5 hours). Within the lag time, not more than 10% of the GLP-1 receptor agonist is released. After the lag time, more than 75% of the GLP-1 receptor agonist is released in simulated intestinal fluid at pH between 6 and 6.5 within 1 hour. Accordingly, it is preferred that the solid oral pharmaceutical composition has a dissolution profile, as determined by the dissolution method according to USP, with less than 5% of the GLP-1 receptor agonist released within 1 hour in simulated gastric fluid, followed by dissolution in simulated intestinal fluid at pH between 6 and 6.5 with a lag time of at least 1 hour, whereby not more than 10% of the GLP-1 receptor agonist is released within the lag time, and whereby after the lag time more than 75% of the GLP-1 receptor agonist is released in simulated intestinal fluid at pH between 6 and 6.5 within 1 hour. As explained above, the dissolution profile should be determined in accordance with the US Pharmacopeia (USP, preferably in the version as of Sep. 1, 2020); alternatively, however, the dissolution profile may also be determined using the modified version of the USP dissolution method described herein below in Example 7 or 26. Exemplary solid oral pharmaceutical compositions according to the present invention having such a dissolution profile are described below in the examples section.

It is furthermore preferred that the solid oral pharmaceutical composition according to the invention shows a release of less than 5% (preferably less than 3%, more preferably less than 1%, even more preferably no release) of the GLP-1 receptor agonist in simulated fed state gastric media, such as e.g. FEDGAS pH 6 (see Example 29), for a period of at least 1 hour, preferably at least 2 hours, more preferably at least 3 hours, most preferably at least 4 hours.

Further preferred examples of solid oral pharmaceutical compositions according to the invention include compositions having a first coating comprising (or consisting of) HPMC, and a second coating (which is exterior to the first coating) comprising (or consisting of) Eudragit FL 30 D-55. In particular, it is preferred that the coating with Eudragit FL 30 D-55 has a thickness leading to a weight gain of at least 45% (w/w) relating to the weight of the first coating with HPMC (e.g., an empty HPMC capsule). Alternatively, it is preferred that the coating with Eudragit FL 30 D-55 has a thickness leading to a weight gain of at least 30 mg, more preferably at least 40 mg, even more preferably at least 50 mg, even more preferably at least 100 mg, per dosage form (e.g., capsule or tablet). Also preferred is a solid oral pharmaceutical composition in the form of a tablet, having a coating comprising (or consisting of) Eudragit FL 30 D-55 with a thickness leading to a weight gain of at least 10% (w/w) (relating to the weight of the tablet before coating with Eudragit FL 30 D-55).

Moreover, also preferred is a solid oral pharmaceutical composition according to the invention, which is in the form of a tablet, having a coating comprising Eudragit NM30D with a coating weight gain of at least 10% (w/w) (e.g., at least 15% (w/w), at least 20% (w/w), or at least 25% (w/w)) in relation to the weight of the uncoated tablet (i.e., before the application of the coating comprising Eudragit NM30D). In the state of the art, it had been assumed that increasing coating weight gains on tablets would decrease the oral bioavailability of GLP-1 peptides (WO 2016/120378, page 61). Surprisingly, however, the inventors found that increasing the coating weight gain with coatings according to the present invention allows to further increase the oral bioavailability of the GLP-1 receptor agonist, especially under fed conditions.

Typically, a physician will determine the actual dosage of the GLP-1 receptor agonist which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy. The precise dose will ultimately be at the discretion of the attendant physician or veterinarian.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease as used herein is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease as used herein is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a peptide or protein drug according to the invention before any clinical and/or pathological symptoms are diagnosed or determined or can be diagnosed or determined by the attending physician.

The terms “peptide” and “protein” are used herein interchangeably and refer to a polymer of two or more amino acids linked via amide bonds that are formed between an amino group of one amino acid and a carboxyl group of another amino acid. The amino acids comprised in the peptide or protein, which are also referred to as amino acid residues, may be selected from the 20 standard proteinogenic α-amino acids (i.e., Ala, Arg, Asn, Asp, Cys, Glu, Gln, Gly, His, Ile, Leu, Lys, Met, Phe, Pro, Ser, Thr, Trp, Tyr, and Val) but also from non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isoglutamine, or statine) and δ-amino acids. Preferably, the amino acid residues comprised in the peptide or protein are selected from α-amino acids, more preferably from the 20 standard proteinogenic α-amino acids (which can be present as the L-isomer or the D-isomer, and are preferably all present as the L-isomer). The peptide or protein may be unmodified or may be modified, e.g., at its N-terminus, at its C-terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., the attachment of any of the protecting groups described for the corresponding functional groups in: Wuts P G & Greene T W, Greene's protective groups in organic synthesis, John Wiley & Sons, 2006. Such modifications may also include the covalent attachment of one or more polyethylene glycol (PEG) chains (forming a PEGylated peptide or protein), the glycosylation and/or the acylation with one or more fatty acids (e.g., one or more C₈₋₃₀ alkanoic or alkenoic acids; forming a fatty acid acylated peptide or protein). Moreover, such modified peptides or proteins may also include peptidomimetics, provided that they contain at least two amino acids that are linked via an amide bond (formed between an amino group of one amino acid and a carboxyl group of another amino acid). The amino acid residues comprised in the peptide or protein may, e.g., be present as a linear molecular chain (forming a linear peptide or protein) or may form one or more rings (corresponding to a cyclic peptide or protein). The peptide or protein may also form oligomers consisting of two or more identical or different molecules.

The term “amino acid” refers, in particular, to any one of the 20 standard proteinogenic α-amino acids (i.e., Ala, Arg, Asn, Asp, Cys, Glu, Gln, Gly, His, Ile, Leu, Lys, Met, Phe, Pro, Ser, Thr, Trp, Tyr, and Val) but also to non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isoglutamine, or statine) and/or δ-amino acids as well as any other compound comprising at least one carboxylic acid group and at least one amino group. Unless defined otherwise, an “amino acid” preferably refers to an α-amino acid, more preferably to any one of the 20 standard proteinogenic α-amino acids (which can be present as the L-isomer or the D-isomer, and are preferably present as the L-isomer).

The term “dissolves” as used herein preferably refers to a state in which the layer whose dissolution is to be determined has been sufficiently dissolved by a solution having the specified pH-value such that it becomes permeable (in particular for the GLP-1 receptor agonist, such as exenatide). Whether a given layer dissolves, can be determined by using a dissolution apparatus 1 as described in United States Pharmacopeia (USP) General Chapter <711> Dissolution.

The term “surrounding” is used herein synonymously with “covering” or “completely covering”.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, if a component of a composition is indicated to be “optional”, the invention specifically relates to both possibilities, i.e., that the corresponding component is present (contained in the composition) or that the corresponding component is absent from the composition.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

As used herein, the term “about” refers to ±10% of the indicated numerical value, preferably to ±5% of the indicated numerical value, and in particular to the exact numerical value indicated.

Unless specifically indicated otherwise, all properties and parameters referred to herein (including, e.g., any amounts/concentrations indicated in “mg/ml” or in “% (v/v)”, and any pH values) are preferably to be determined at standard ambient temperature and pressure conditions, particularly at a temperature of 25° C. (298.15 K) and at an absolute pressure of 101.325 kPa (1 atm).

Furthermore, it is to be understood that the present invention specifically relates to each and every combination of features and embodiments described herein, including any combination of general and/or preferred features/embodiments. In particular, the invention specifically relates to all combinations of preferred features described herein.

In this specification, a number of documents including patents, patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention is also described by the following illustrative figures:

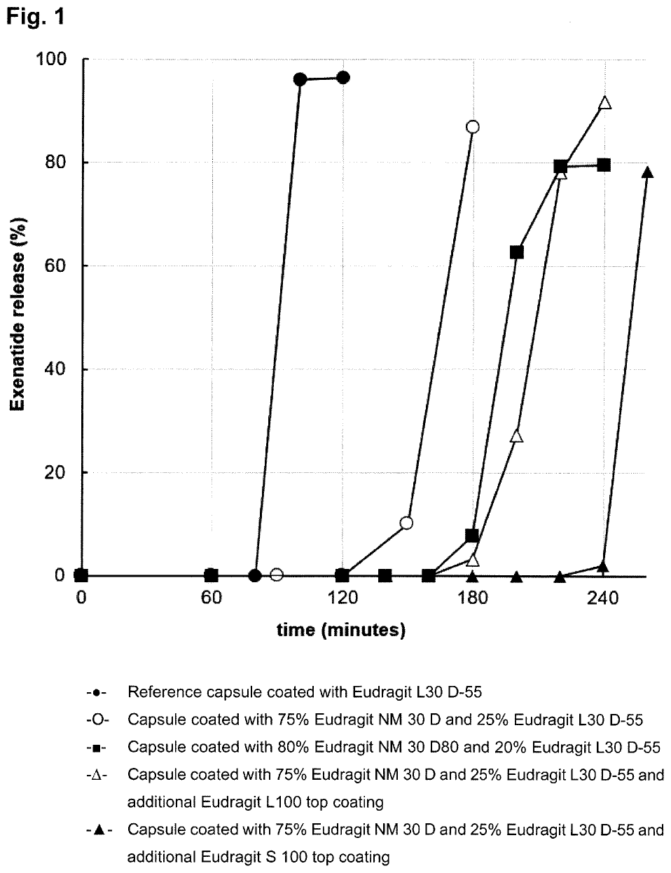

FIG. 1: Dissolution of coated capsules comprising the GLP-1 agonist peptide exenatide and sodium caprate (see Example 7).

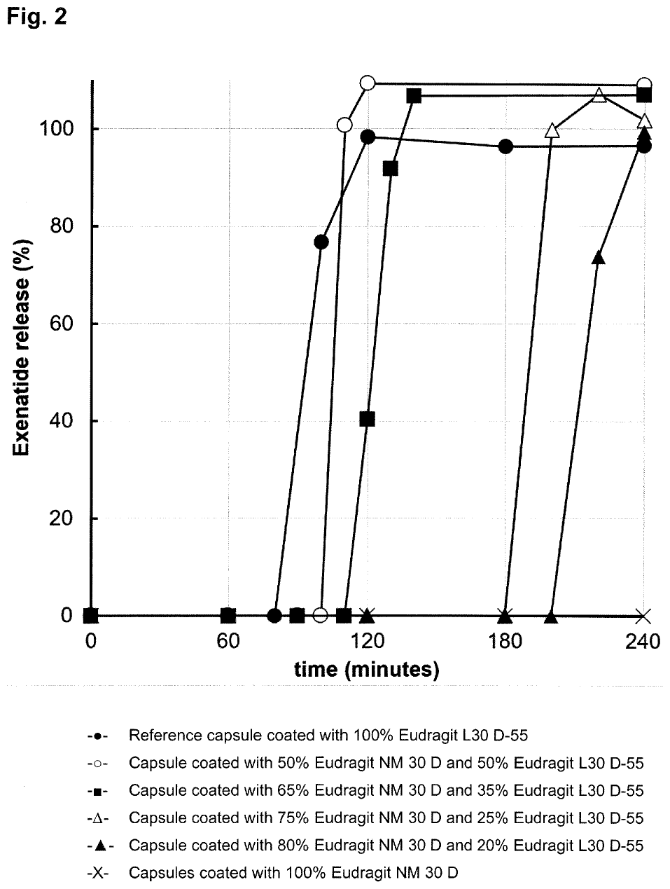

FIG. 2: Dissolution of exenatide from capsules coated with different ratios of Eudragit L30D-55 and Eudragit NM30D (see Example 26).

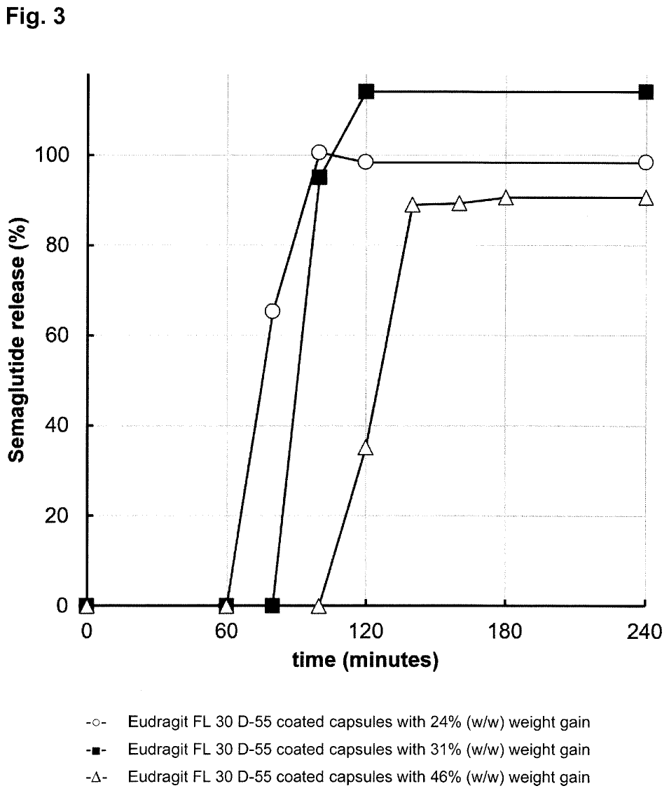

FIG. 3: Dissolution of semaglutide from Eudragit FL 30 D-55 coated capsules with increasing coating weight gains (see Example 27).

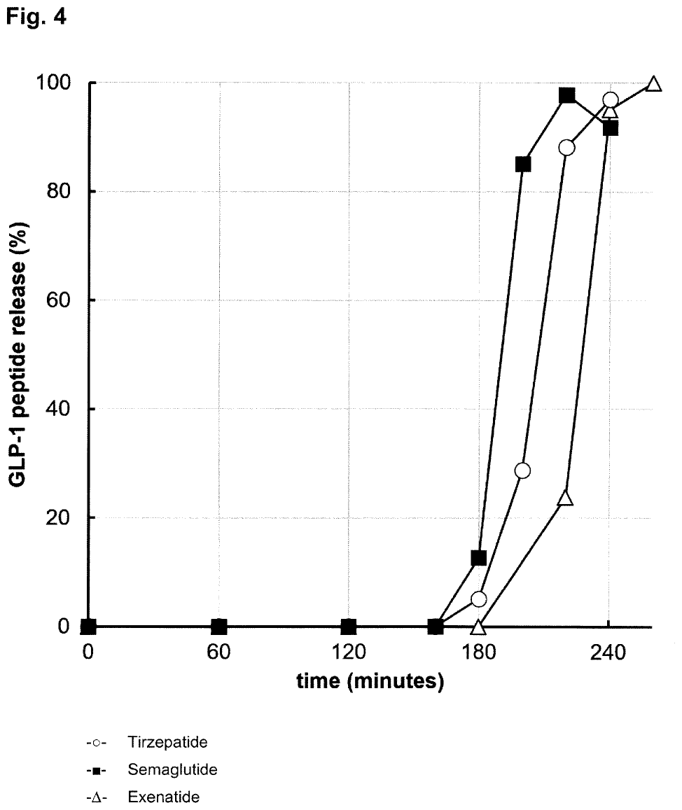

FIG. 4: Dissolution of GLP-1 agonists (tirzepatide, semaglutide or exenatide) from capsules coated with Eudragit NM30D (80%) and Eudragit L30 D-55 (20%) (see Example 28).

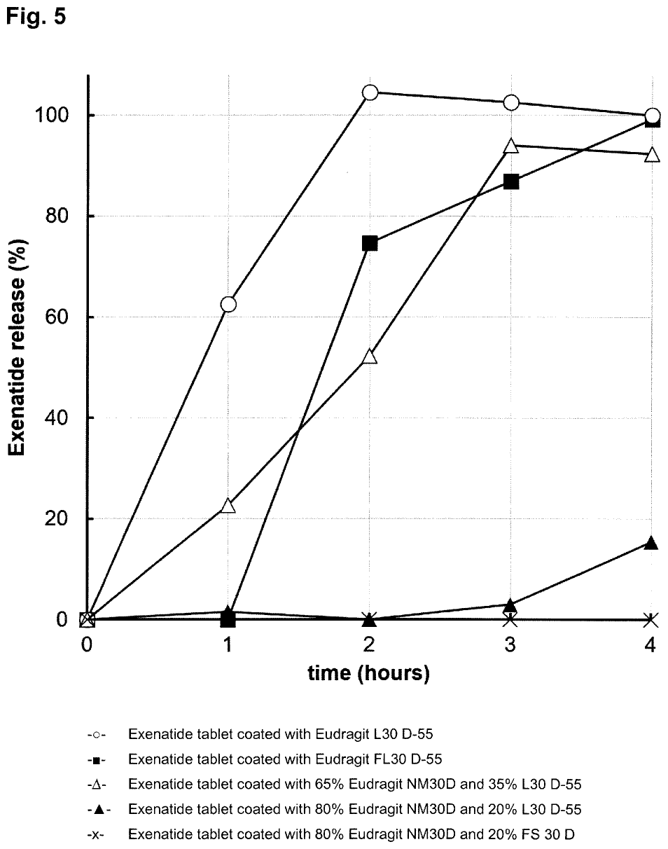

FIG. 5: Stability of enteric tablets comprising GLP-1 agonists in Fed State Simulated Gastric Fluid (FEDGAS) (see Example 29).

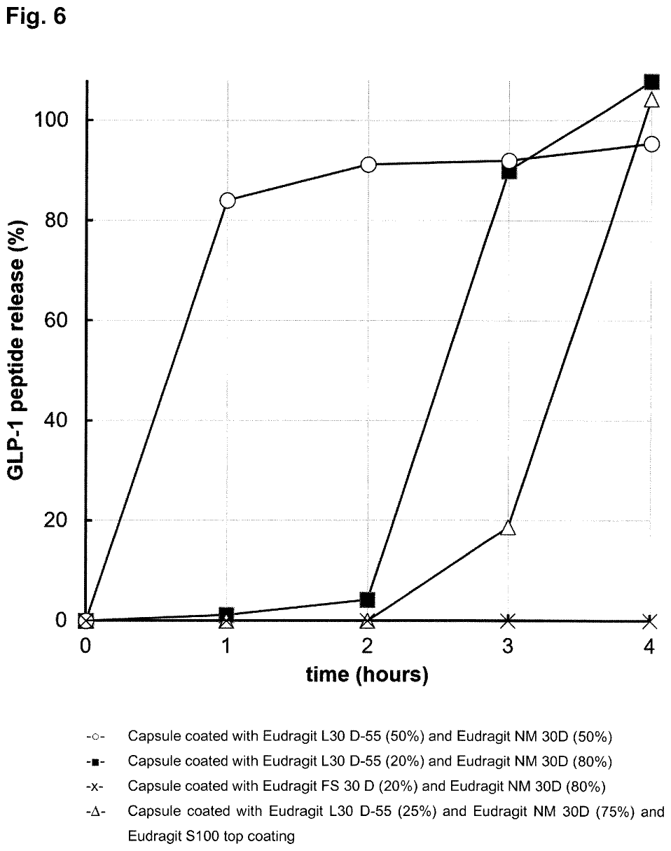

FIG. 6: Stability of enteric capsules comprising GLP-1 agonists in FEDGAS (see Example 29).

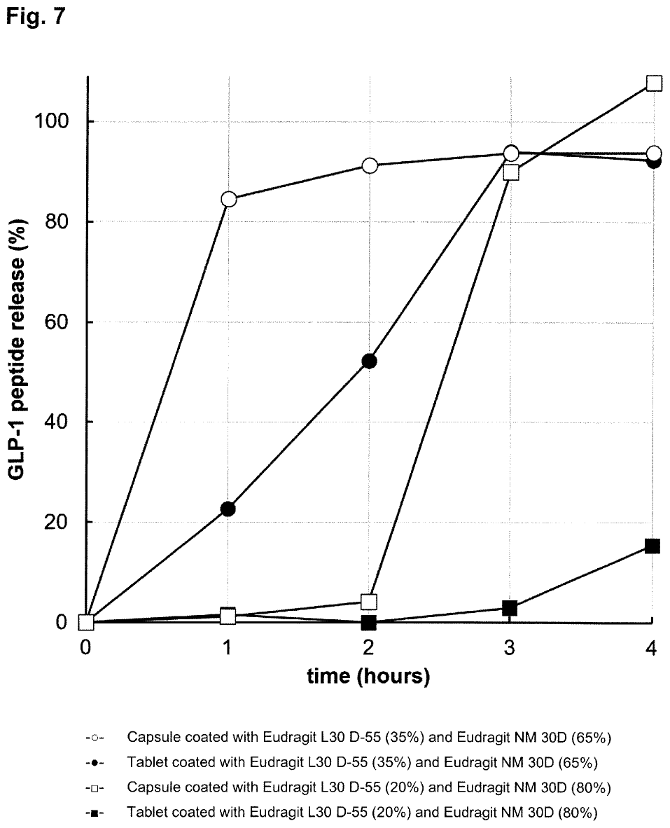

FIG. 7: Stability of enteric capsules compared with enteric tablets in FEDGAS (see Example 29).

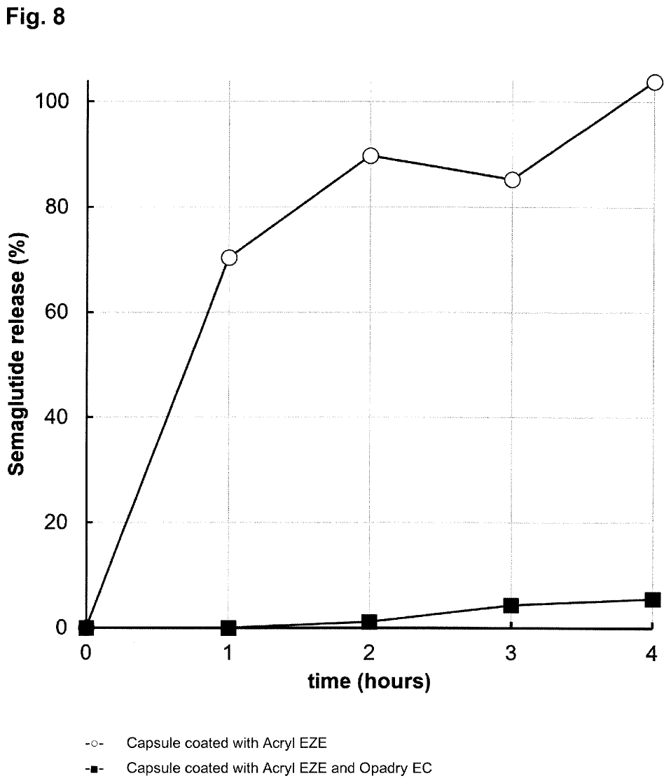

FIG. 8: Stability of enteric capsules comprising GLP-1 agonists in FEDGAS (see Example 30).

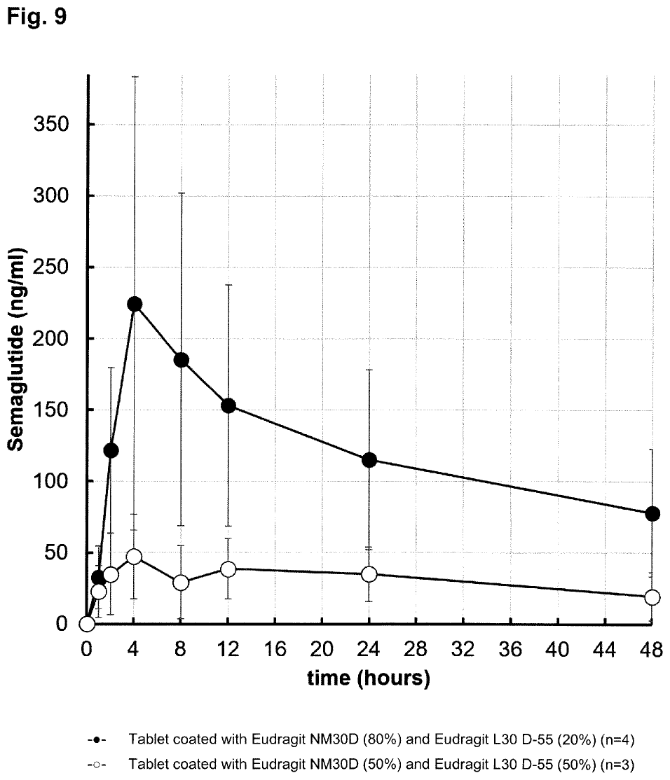

FIG. 9: Pharmacokinetic profile of semaglutide after oral administration of enteric tablets to beagle dogs in fasted stage (see Example 31).

In accordance with the detailed description above, the present invention particularly relates to the following items:
1. A solid oral pharmaceutical composition comprising:
   (i) a core comprising a GLP-1 receptor agonist, and
   (ii) a first coating, wherein the first coating comprises
      (ii-1) a copolymer (A) in combination with
      (ii-2) a copolymer (B) and/or a copolymer (C) and/or a copolymer (D);
      wherein the copolymer (A) comprises:
         (a) 20 to 90 mol-% ethyl acrylate repeating units, and
         (b) 10 to 80 mol-% methyl methacrylate repeating units;
      wherein the copolymer (B), if present, comprises:
         (a) 25 to 75 mol-% methacrylic acid repeating units, and
         (b) 25 to 75 mol-% ethyl acrylate repeating units;
      wherein the copolymer (C), if present, comprises:
         (a) 25 to 60 mol-% methacrylic acid repeating units, and
         (b) 40 to 75 mol-% methyl methacrylate repeating units;
      wherein the copolymer (D), if present, comprises:
         (a) 5 to 20 mol-% methacrylic acid repeating units, and
         (b) 20 to 40 mol-% methyl methacrylate repeating units, and
         (c) 60 to 75 mol-% methyl acrylate repeating units.
2. The solid oral pharmaceutical composition according to item 1, wherein the first coating comprises
   (ii-1) a copolymer (A) in combination with
   (ii-2) a copolymer (B) and/or a copolymer (C).
3. The solid oral pharmaceutical composition according to item 1 or 2, wherein the copolymer (A) in the first coating comprises 60 to 75 mol-% ethyl acrylate repeating units, and 25 to 40 mol-% methyl methacrylate repeating units.
4. The solid oral pharmaceutical composition according to any one of items 1 to 3, wherein the copolymer (A) in the first coating comprises ethyl acrylate repeating units and methyl methacrylate repeating units in a molar ratio of 2:1.
5. The solid oral pharmaceutical composition according to any one of items 1 to 4, wherein the copolymer (A) in the first coating comprises not more than 3 mol-% methyl acrylate repeating units, preferably wherein the copolymer (A) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
6. The solid oral pharmaceutical composition according to any one of items 1 to 5, wherein the copolymer (A) in the first coating consists of ethyl acrylate repeating units and methyl methacrylate repeating units.
7. The solid oral pharmaceutical composition according to any one of items 1 to 6, wherein the copolymer (A) in the first coating is a neutral non-ionic copolymer.
8. The solid oral pharmaceutical composition according to item 1 or 2, wherein the copolymer (A) in the first coating further comprises 0.5 to 20 mol-%, preferably 1 to 15 mol-%, 2-(trimethylammonio)ethyl methacrylate chloride repeating units.
9. The solid oral pharmaceutical composition according to any one of items 1, 2 and 8, wherein the copolymer (A) in the first coating comprises 25 to 39 mol-% ethyl acrylate repeating units, 60 to 74 mol-% methyl methacrylate repeating units, and 1 to 15 mol-% 2-(trimethylammonio)ethyl methacrylate chloride repeating units.
10. The solid oral pharmaceutical composition according to any one of items 1, 2, 8 and 9, wherein the copolymer (A) in the first coating comprises ethyl acrylate repeating units, methyl methacrylate repeating units, and 2-(trimethylammonio)ethyl methacrylate chloride repeating units in a molar ratio of 1:2:0.1 or 1:2:0.2.
11. The solid oral pharmaceutical composition according to any one of items 1, 2 and 8 to 10, wherein the copolymer (A) in the first coating comprises not more than 3 mol-% methyl acrylate repeating units, preferably wherein the copolymer (A) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
12. The solid oral pharmaceutical composition according to any one of items 1, 2 and 8 to 11, wherein the copolymer (A) in the first coating consists of ethyl acrylate repeating units, methyl methacrylate repeating units, and 2-(trimethylammonio)ethyl methacrylate chloride repeating units.
13. The solid oral pharmaceutical composition according to any one of items 1, 2 and 8 to 12, wherein the copolymer (A) in the first coating is a cationic copolymer.
14. The solid oral pharmaceutical composition according to any one of items 1 to 13, wherein the copolymer (B) in the first coating comprises 45 to 55 mol-% methacrylic acid repeating units, and 45 to 55 mol-% ethyl acrylate repeating units.
15. The solid oral pharmaceutical composition according to any one of items 1 to 14, wherein the copolymer (B) in the first coating comprises methacrylic acid repeating units and ethyl acrylate repeating units in a molar ratio of 1:1.
16. The solid oral pharmaceutical composition according to any one of items 1 to 15, wherein the copolymer (B) in the first coating comprises not more than 3 mol-% methyl acrylate repeating units, preferably wherein the copolymer (B) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
17. The solid oral pharmaceutical composition according to any one of items 1 to 16, wherein the copolymer (8) in the first coating consists of methacrylic acid repeating units and ethyl acrylate repeating units.
18. The solid oral pharmaceutical composition according to any one of items 1 to 17, wherein the copolymer (C) in the first coating comprises 45 to 55 mol-% methacrylic acid repeating units, and 45 to 55 mol-% methyl methacrylate repeating units.
19. The solid oral pharmaceutical composition according to any one of items 1 to 18, wherein the copolymer (C) in the first coating comprises methacrylic acid repeating units and methyl methacrylate repeating units in a molar ratio of 1:1.
20. The solid oral pharmaceutical composition according to any one of items 1 to 19, wherein the copolymer (C) in the first coating comprises not more than 3 mol-% methyl acrylate repeating units, preferably wherein the copolymer (C) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
21. The solid oral pharmaceutical composition according to any one of items 1 to 20, wherein the copolymer (C) in the first coating consists of methacrylic acid repeating units and methyl methacrylate repeating units.
22. The solid oral pharmaceutical composition according to any one of items 1 to 17, wherein the copolymer (C) in the first coating comprises 25 to 40 mol-% methacrylic acid repeating units, and 60 to 75 mol-% methyl methacrylate repeating units.
23. The solid oral pharmaceutical composition according to any one of items 1 to 17 and 22, wherein the copolymer (C) in the first coating comprises methacrylic acid repeating units and methyl methacrylate repeating units in a molar ratio of 1:2.
24. The solid oral pharmaceutical composition according to any one of items 1 to 17, 22 and 23, wherein the copolymer (C) in the first coating comprises not more than 3 mol-% methyl acrylate repeating units, preferably wherein the copolymer (C) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
25. The solid oral pharmaceutical composition according to any one of items 1 to 17 and 22 to 24, wherein the copolymer (C) in the first coating consists of methacrylic acid repeating units and methyl methacrylate repeating units.
26. The solid oral pharmaceutical composition according to any one of items 1 to 25, wherein the copolymer (C) in the first coating is obtained from an aqueous dispersion of copolymer (C).
27. The solid oral pharmaceutical composition according to any one of items 1 to 26, wherein the copolymer (D) in the first coating comprises 7 to 13 mol-% methacrylic acid repeating units, 25 to 31 mol-% methyl methacrylate repeating units, and 62 to 68 mol-% methyl acrylate repeating units.
28. The solid oral pharmaceutical composition according to any one of items 1 to 27, wherein the copolymer (D) in the first coating comprises methacrylic acid repeating units, methyl methacrylate repeating units, and methyl acrylate repeating units in a molar ratio of 1:3:7.
29. The solid oral pharmaceutical composition according to any one of items 1 to 28, wherein the copolymer (D) in the first coating consists of methacrylic acid repeating units, methyl methacrylate repeating units, and methyl acrylate repeating units.
30. The solid oral pharmaceutical composition according to any one of items 1 to 29, wherein the content of the copolymer (A) in the first coating is at least 25% (w/w), preferably at least 50% (w/w), more preferably at least 75% (w/w), even more preferably at least 80% (w/w), yet even more preferably at least 90% (w/w), in relation to the total weight of the first coating.
31. The solid oral pharmaceutical composition according to any one of items 1 to 30, wherein the first coating comprises the copolymer (A) and the copolymer (B), wherein the content of the copolymer (A) in the first coating is at least 25% (w/w), preferably at least 50% (w/w), more preferably at least 75% (w/w), even more preferably at least 80% (w/w), yet even more preferably at least 90% (w/w), in relation to the total weight of the copolymer (A) and the copolymer (B) in the first coating.
32. The solid oral pharmaceutical composition according to any one of items 1 to 30, wherein the first coating comprises the copolymer (A) and the copolymer (C), wherein the copolymer (C) is as defined in any one of items 18 to 21.
33. The solid oral pharmaceutical composition according to any one of items 1 to 30, wherein the first coating comprises the copolymer (A) and the copolymer (C), wherein the copolymer (C) is as defined in any one of items 22 to 26.
34. The solid oral pharmaceutical composition according to any one of items 1 to 33, wherein the first coating further comprises one or more polymers selected from ethyl cellulose, hydroxypropyl methylcellulose (HPMC), and polyvinyl acetate, either in addition to the copolymer (A) or instead of it.
35. The solid oral pharmaceutical composition according to any one of items 1, 3 to 26 and 30 to 34, wherein the first coating does not contain any copolymer (D) as defined in any one of items 1 or 27 to 29.
36. The solid oral pharmaceutical composition according to any one of items 1 to 35, wherein the first coating dissolves at a pH in the range of 5 to 7, preferably at a pH in the range of 5.5 to 6.5, more preferably at a pH in the range of 5.5 to 6.0.
37. The solid oral pharmaceutical composition according to any one of items 1 to 36, further comprising:
   (iii) a second coating which is exterior to the first coating, wherein the second coating comprises a copolymer (C);
      wherein the copolymer (C) comprises:
         (a) 25 to 60 mol-% methacrylic acid repeating units, and
         (b) 40 to 75 mol-% methyl methacrylate repeating units.
38. The solid oral pharmaceutical composition according to item 37, wherein the copolymer (C) in the second coating comprises 45 to 55 mol-% methacrylic acid repeating units, and 45 to 55 mol-% methyl methacrylate repeating units.
39. The solid oral pharmaceutical composition according to items 37 or 38, wherein the copolymer (C) in the second coating comprises methacrylic acid repeating units and methyl methacrylate repeating units in a molar ratio of 1:1.
40. The solid oral pharmaceutical composition according to any one of items 37 to 39, wherein the copolymer (C) in the second coating comprises not more than 3 mol-% methyl acrylate repeating units, preferably wherein the copolymer (C) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
41. The solid oral pharmaceutical composition according to item 37, wherein the copolymer (C) in the second coating consists of methacrylic acid repeating units and methyl methacrylate repeating units.
42. The solid oral pharmaceutical composition according to items 37 or 41, wherein the copolymer (C) in the second coating comprises 25 to 40 mol-% methacrylic acid repeating units, and 60 to 75 mol-% methyl methacrylate repeating units.
43. The solid oral pharmaceutical composition according to any one of items 37, 41 and 42, wherein the copolymer (C) in the second coating comprises methacrylic acid repeating units and methyl methacrylate repeating units in a molar ratio of 1:2.
44. The solid oral pharmaceutical composition according to any one of items 37 and 41 to 43, wherein the copolymer (C) in the second coating comprises not more than 3 mol-% methyl acrylate repeating units, preferably wherein the copolymer (C) comprises not more than 1 mol-%, more preferably not more than 0.5 mol-%, even more preferably not more than 0.1 mol-%, yet even more preferably not more than 0.01 mol-%, still more preferably 0 mol-% methyl acrylate repeating units.
45. The solid oral pharmaceutical composition according to any one of items 37 and 41 to 44, wherein the copolymer (C) in the second coating consists of methacrylic acid repeating units and methyl methacrylate repeating units.
46. The solid oral pharmaceutical composition according to any one of items 1 to 45, wherein the second coating dissolves at a pH in the range of 5 to 7, preferably at a pH in the range of 5.5 to 6.5, more preferably at a pH in the range of 5.5 to 6.0.
47. The solid oral pharmaceutical composition according to any one of items 1 to 46, wherein the GLP-1 receptor agonist is a peptide.
48. The solid oral pharmaceutical composition according to any one of items 1 to 47, wherein the GLP-1 receptor agonist is a peptide consisting of the sequence:

(SEQ ID NO: 1)
Xaa⁷-Xaa⁸-Glu-Gly-Thr-Xaa¹²-Thr-Ser-Asp-Xaa¹⁶-Ser-
Xaa¹⁸-Xaa¹⁹-Xaa²⁰-Glu-Xaa²²-Xaa²³-Xaa²⁴-Xaa²⁵-Xaa²⁶-
Lys-Phe-Ile-Xaa³⁰-Xaa³¹-Leu-Val-Xaa³⁴-Xaa³⁵-Xaa³⁶-
Xaa³⁷-Xaa³⁸-Xaa³⁹,

wherein:
   Xaa⁷ is L-histidine, imidazopropionyl, α-hydroxy-histidine, D-histidine, deamino-histidine, 2-amino-histidine, β-hydroxy-histidine, homohistidine, Nα-acetyl-histidine, Na-formyl-histidine, α-fluoromethyl-histidine, α-methyl-histidine, 3-pyridylalanine, 2-pyridylalanine, or 4-pyridylalanine;
   Xaa⁸ is Ala, Gly, Val, Leu, Ile, Thr, Ser, Lys, α-aminoisobutyric acid, (1-aminocyclopropyl)carboxylic acid, (1-aminocyclobutyl)carboxylic acid, (1-aminocyclopentyl)carboxylic acid, (1-aminocyclohexyl)carboxylic acid, (1-aminocycloheptyl)carboxylic acid, or (1-aminocyclooctyl)carboxylic acid;
   Xaa¹² is Lys or Phe;
   Xaa¹⁸ is Val or Leu;
   Xaa¹⁸ is Ser, Arg, Asn, Gln, or Glu;
   Xaa¹⁹ is Tyr or Gln;
   Xaa²⁰ is Leu, Lys, or Met;
   Xaa²² is Gly, Glu, Lys, or α-aminoisobutyric acid;
   Xaa²³ is Gln, Glu, or Arg;
   Xaa²⁴ is Ala or Lys;
   Xaa²⁵ is Ala or Val;
   Xaa²⁶ is Val, His, Lys or Arg;
   Xaa³⁰ is Ala, Glu, or Arg;
   Xaa³¹ is Trp or His;
   Xaa³⁴ is Glu, Asn, Gly, Gln, or Arg;
   Xaa³⁵ is Gly, α-aminoisobutyric acid, or absent;
   Xaa³⁸ is Arg, Gly, Lys, or absent;
   Xaa³⁷ is Gly, Ala, Glu, Pro, Lys, Arg, or absent;
   Xaa³⁸ is Ser, Gly, Ala, Glu, Gln, Pro, Arg, or absent; and
   Xaa³⁹ is Gly or absent.
49. The solid oral pharmaceutical composition according to any one of items 1 to 48, wherein the GLP-1 receptor agonist is selected from semaglutide, liraglutide, exenatide, albiglutide, dulaglutide, lixisenatide, taspoglutide, langlenatide, beinaglutide, efpeglenatide, GLP-1(7-37), GLP-1(7-36)NH₂, and oxyntomodulin; preferably wherein the GLP-1 receptor agonist is selected from semaglutide, liraglutide, exenatide, albiglutide, dulaglutide, and lixisenatide.
50. The solid oral pharmaceutical composition according to any one of items 1 to 49, wherein the GLP-1 receptor agonist is a dual GLP-1 receptor/glucagon receptor agonist, a dual GLP-1 receptor/GIP receptor agonist, or a triple GLP-1 receptor/GIP receptor/glucagon receptor agonist.
51. The solid oral pharmaceutical composition according to any one of items 1 to 50, wherein the core further comprises one or more permeation enhancers.
52. The solid oral pharmaceutical composition according to any one of items 1 to 51, wherein the solid oral pharmaceutical composition is an oral dosage form.
53. The solid oral pharmaceutical composition according to any one of items 1 to 52, wherein the solid oral pharmaceutical composition is in the form of a capsule.
54. The solid oral pharmaceutical composition according to any one of items 1 to 52, wherein the solid oral pharmaceutical composition is in the form of a tablet.
55. The solid oral pharmaceutical composition according to any one of items 1 to 54, wherein the core is in the form of a multiparticulate, preferably wherein the core is in the form of a granulate or pellets.
56. The solid oral pharmaceutical composition according to any one of items 1 to 55, wherein the solid oral pharmaceutical composition has a dissolution profile, as determined by the dissolution method according to USP, with less than 5% of the GLP-1 receptor agonist released within 2 hours in simulated gastric fluid, followed by dissolution in simulated intestinal fluid at pH between 6 and 6.5 with a lag time of at least 1 hour, whereby not more than 10% of the GLP-1 receptor agonist is released within the lag time, and whereby after the lag time more than 75% of the GLP-1 receptor agonist is released in simulated intestinal fluid at pH between 6 and 6.5 within 1 hour.
57. The solid oral pharmaceutical composition according to any one of items 1 to 56 for use in therapy.
58. The solid oral pharmaceutical composition according to any one of items 1 to 56 for use in the treatment or prevention of diabetes, obesity, non-alcoholic fatty liver disease, non-alcoholic steatohepatitis (NASH) or a cardiovascular disease.
59. The solid oral pharmaceutical composition according to any one of items 1 to 56 for use in the treatment or prevention of type 2 diabetes.
60. The solid oral pharmaceutical composition for use according to any one of items 57 to 59, wherein the solid oral pharmaceutical composition is to be administered orally.
61. Use of the solid oral pharmaceutical composition according to any one of items 1 to 56 for the manufacture of a medicament for the treatment or prevention of diabetes, obesity, non-alcoholic fatty liver disease, non-alcoholic steatohepatitis (NASH) or a cardiovascular disease.
62. Use of the solid oral pharmaceutical composition according to any one of items 1 to 56 for the manufacture of a medicament for the treatment or prevention of type 2 diabetes.
63. A method of treating or preventing diabetes, obesity, non-alcoholic fatty liver disease, non-alcoholic steatohepatitis (NASH) or a cardiovascular disease in a subject in need thereof, the method comprising orally administering a therapeutically effective amount of the solid oral pharmaceutical composition according to any one of items 1 to 56 to the subject.
64. A method of treating or preventing type 2 diabetes in a subject in need thereof, the method comprising orally administering a therapeutically effective amount of the solid oral pharmaceutical composition according to any one of items 1 to 56 to the subject.
65. A method of delivering a GLP-1 receptor agonist to a subject in need thereof, the method comprising orally administering the solid oral pharmaceutical composition according to any one of items 1 to 56 to the subject.

The invention will now be described by reference to the following examples which are merely illustrative and are not to be construed as a limitation of the scope of the present invention.

### EXAMPLES

Materials

The following materials were used in the experimental examples:

All Eudragit polymers and PlasAcryl were obtained from Evonik (Germany). All other chemicals were obtained from Sigma Aldrich (Austria) or VWR (Austria).

Eudragit NM 30 D:

Aqueous dispersion of a neutral copolymer based on ethyl acrylate and methyl methacrylate. Chemical/IUPAC name: Poly(ethyl acrylate-co-methyl methacrylate) 2:1.

Eudragit® L 30 D-55:

Aqueous dispersion of an anionic copolymer based on methacrylic acid and ethyl acrylate. The ratio of the free carboxyl groups to the ester groups is approximately 1:1.

Eudragit S 100 Anionic copolymer based on methacrylic acid and methyl methacrylate; chemical name:

Poly(methacrylic acid-co-methyl methacrylate) 1:2.

Eudragit L 100

Anionic copolymer based on methacrylic acid and methyl methacrylate; chemical name: Poly(methacrylic acid-co-methyl methacrylate) 1:1.

Eudragit FL 30 D-55

Ready to use combination polymer product of 2 Eudragit Polymers (Eudragit L30 D-55 (25%) and Eudragit NM 30 D (75%)).

Eudragit FS 30 D

Aqueous dispersion of an anionic copolymer based on methylacrylate, methyl methacrylate and methacrylic acid. Poly(methylacrylat-co-methyl-methacrylat-co-methacrylic acid) 7:3:1.

Opadry EC (from Colorcon)

Opadry EC is an organic ready to use coating product from Colorcon comprising ethylcellulose in solution.

Acryl Eze (from Colorcon)

Acryl-Eze is a fully formulated coating system of Eudragit® L100-55 (a copolymer of methacrylic acid and ethyl acrylate (1:1 ratio)).

### Example 1: Coating of HPMC Capsules with a Combination of 80% Eudragit NM 30 D and 20% Eudragit L 30 D-55

Preparation of the Eudragit dispersions: The pH of 109 g of Eudragit NM 30 D was adjusted to about 3 with 1.3 ml of a 20% (w/w) citric acid solution. 67 g of distilled water were added under conventional stirring. 28 g of Eudragit L 30 D-55 were added followed by the addition of 2 g of Plasacryl T20. The final composition was stirred for 15 minutes with a conventional stirrer.

Coating of HPMC capsules: Empty size 1 HPMC capsules, each weighing about 70 mg, were coated with the Eudragit dispersion using a Glatt GC1 lab coater to a final weight of 95 mg, 119 mg and 125 mg.

### Example 2: Coating of HPMC Capsules with a Combination of 75% Eudragit NM 30 D and 25% Eudragit L 30 D-55

Preparation of the Eudragit dispersions: The pH of 100 g of Eudragit NM 30 D was adjusted to about 3 with 1.3 ml of a 20% (w/w) citric acid solution. 67 g of distilled water were added under conventional stirring. 33.3 g of Eudragit L 30 D-55 were then added, followed by the addition of 2 g of Plasacryl T20. The final composition was stirred for 15 minutes with a conventional stirrer.

Coating of HPMC capsules: Empty size 1 HPMC capsules, each weighing about 70 mg, were coated with the Eudragit dispersion using a Glatt GC1 lab coater to a final weight of 115 mg.

### Example 3: Eudragit L 100 Top-Coating on Coated Capsules from Example 2

Preparation of the dispersions: 20 g of Eudragit L 100 were dispersed in distilled 100 ml water. 11.2 ml of 1 N NH₃ were added slowly under continuous stirring. Stirring was continued for 60 minutes. 10 g of triethylcitrate (TEC) were then added and stirring continued for another 60 minutes. Separately, 10 g of talc were homogenized in 50 g of water with a high shear mixer (Ultra Turrax). Both dispersions were combined under continuous stirring with a conventional stirrer.

Top-coating of capsules: Capsules from Example 2 (with a weight of 115 mg) were top-coated with the Eudragit L 100 dispersion obtained above using a Glatt GC1 lab coater to a final weight of 130 mg.

### Example 4: Eudragit S 100 Top-Coating on Coated Capsules from Example 2

Preparation of the dispersions: 19.9 g of Eudragit S 100 were dispersed in 100 ml distilled water. 13.5 ml of 1 N NH₃ was added slowly under continuous stirring. Stirring was continued for 60 minutes. Then 10 g of Triethylcitrate (TEC) were added and stirring continued for another 60 minutes. Separately 10 g of talc were homogenized in 50 g of water with a high shear mixer (Ultra Turrax). Both dispersions were combined under continuous stirring with a conventional stirrer.

Top-coating of capsules: Capsules from Example 2 (with a weight of 115 mg) were top-coated with the Eudragit S 100 dispersion obtained above using a Glatt GC1 lab coater to a final weight of 132 mg.

### Example 5: Enteric Coating of HPMC Capsules with Eudragit S100 Redisperion Comprising 50% TEC

Preparation of the Eudraqit aqueous dispersion: 99.4 g of EUDRAGIT® S 100 was added slowly into 500 ml of water and stirred for approx. 5 minutes with a conventional stirrer. 67.5 g of 1N NH₃ were then added slowly into the EUDRAGIT® suspension and stirred for about 60 minutes. Next 49.7 g of triethyl citrate (TEC) were added into the EUDRAGIT® suspension and stirred again for 60 minutes. 49.7 g of talc were homogenized in 233.7 g of water for 10 minutes with a high shear mixer (e.g. Ultra Turrax). Then the talc suspension was poured into the EUDRAGIT® dispersion while stirring with a conventional stirrer. Finally, the spray suspension was passed through a 0.5 mm sieve and stirred continuously.

Coating of HPMC capsules: HPMC capsules were coated with the Eudragit aqueous dispersion using a Glatt GC1 lab coater to a weight gain of 18%, 60% and 64% (calculated based on weight of empty capsules).

### Example 6: Enteric Coating of HPMC Capsules with Eudragit S100 Redisperion Comprising 70% TEC

Preparation of the Eudragit aqueous disperion: 99.4 g of EUDRAGIT® S 100 was added slowly into 500 ml of water and stirred for approx. 5 minutes with a conventional stirrer. 67.5 g of 1N NH₃ were then slowly added into the EUDRAGIT® suspension and stirred for about 60 minutes. Next 70 g of triethyl citrate (TEC) were added into the EUDRAGIT® suspension and stirred for again for 60 minutes. 49.7 g of talc were homogenized in 233.7 g of water for 10 minutes with a high shear mixer (e.g. Ultra Turrax). Then the talc suspension was poured into the EUDRAGIT® dispersion while stirring with a conventional stirrer. Finally, the spray suspension was passed through a 0.5 mm sieve and stirred continuously.

Enteric coating of HPMC capsules: HPMC capsules were coated with the Eudragit aqueous dispersion using a Glatt GC1 lab coater to a weight gain of 30%, 50% and 63% (calculated based on weight of empty capsules).

### Example 7: Dissolution Testing of Coated Capsules

Pre-coated capsules were filled with 10 mg exenatide and 200 mg of sodium caprate. Dissolution studies were performed with an Erweka DT light 126, using the basket method at 37° C. and a rotation speed of 75 rpm. A modified version of the United States Pharmacopeia (USP) method was used. The capsules were put into the baskets and the baskets were placed in open blue cap bottles, containing 100 ml of the according dissolution media. The blue cap bottles were in direct contact with water, ensuring a core temperature inside the blue cap bottles of 37° C. (temperature was confirmed prior to starting the test with an external thermometer). Baskets were first placed into simulated gastric fluid according to USP for one hour, then for one hour in simulated intestinal fluid (SIF) according to USP with pH 6.0, then for one hour in SIF according to USP with pH 6.5 and finally in SIF according to USP with pH 6.8. Samples of 1 ml were withdrawn after 60 minutes and at further pre-determined time-points. 40 μl of the samples were injected into a HPLC system using a reversed phase gradient method (water/acetonitrile+0.1% trifluoracetic acid; column: Waters Xselect CSH C18). Sampling was performed until full capsule dissolution, defined by an exenatide release of >75% of the theoretical release value.

The results thus obtained are shown in FIG. 1. These results show that capsules coated according to the present invention demonstrate no drug release at the acid stage, followed by a lag time at intestinal pH levels and then a rather fast release profile.

### Example 8: Coating of HPMC Capsules with Eudragit L100 Redispersion (Reference)

Preparation of the dispersions: 20 g of Eudragit L 100 were dispersed in distilled 100 ml water. 11.2 ml of 1 N NH₃ were added slowly under continuous stirring. Stirring was continued for 60 minutes. 10 g of triethylcitrate (TEC) were then added and stirring continued for another 60 minutes. Separately, 10 g of talc were homogenized in 50 g of water with a high shear mixer (Ultra Turrax). Both dispersions were combined under continuous stirring with a conventional stirrer.

Coating of (reference) capsules: Capsules (HPMC capsules size 0 with a starting weight of 95.3 mg) were coated with the Eudragit L 100 dispersion obtained above using a Glatt GC1 lab coater to a final weight of 137 mg (weight gain of 44%).

### Example 9: Coating of Size 4 HPMC Capsules with a Combination of 80% Eudragit NM 30 D and 20% Eudragit L 30 D-55

Preparation of the Eudragit dispersions: The pH of 109 g of Eudragit NM 30 D was adjusted to about 3 with 1.3 ml of a 20% (w/w) citric acid solution. 67 g of distilled water were added under conventional stirring. 28 g of Eudragit L 30 D-55 was added followed by the addition of 2 g of Plasacryl T20. The final composition was stirred for 15 minutes with a conventional stirrer.

Coating of HPMC capsules: Empty size 4 HPMC capsules, each weighing about 39.5 mg, were coated with the Eudragit dispersion using a Glatt GC1 lab coater to a final weight gain of 54%.

### Example 10: Enteric Coating of Tablets Comprising Exenatide and Sodium Caprate

Preparation of the tablets: A homogenous powder blend with 100 mg of exenatide, 5.000 g of sodium caprate, 1.300 g of sorbitol, 1.300 g of Avicel PH-101 and 100 mg of magnesium stearate was prepared. Mixing was first done in a mortar followed by mixing with a Topitec powder blender. Aliquots of 780 mg were compressed into tablets with a Korsch EK0 single punch tablet press.

Preparation of the Eudragit dispersion: The pH of 109 g of Eudragit NM 30 D was adjusted to about 3 with 1.3 ml of a 20% (w/w) citric acid solution. 67 g of distilled water were added under conventional stirring. 28 g of Eudragit L 30 D-55 was added followed by the addition of 2 g of Plasacryl T20. The final composition was stirred for 15 minutes with a conventional stirrer.

Coating of tablets: Tablets were blended with placebo tablets of comparable weight and dimensions and coated with the Eudragit dispersion obtained above using a Glatt GC1 lab coater to final weight gains between 809 and 816 mg.

### Example 11: Per-Oral Administration of Coated Capsules to Pigs

Evaluation of the pharmacokinetics of single oral semaglutide (10 mg/pig) formulated with two different enteric capsule formulations in pigs.
Species: Sus scrofa domestica
Sex: Male/Female
Weight at start of study: 17.8±3.0 kg

Acclimatization, housing and food:

Pigs were acclimated to the facility for 2 days. Animals were housed with straw and provided with water and standard feed ad libitum from arrival until the completion of the study. Pigs were fasted 6 hours prior and 6 hours after capsule administration. A total of 10 healthy pigs were used in the study. Animals were randomized to one of two experimental groups (n=3-4).

Experiment:

Animals were fasted 6 hours prior dose administration and 6 hours after capsule administration on day 0. On the day of the experiment, intravenous puncture of the Vena cava cranialis/Vena jugularis was performed (21G needle). Baseline blood samples were collected followed by oral administration of a single semaglutide capsule (by using a pill application device) per animal according to the groups and formulations detailed below:

Group 1 (75% Eudragit NM30D, 25% Eudragit L30 D-55 with Eudragit L100 top coat):

Enteric coated capsules according to Example 3 were filled with a homogenous powder blend containing 10 mg semaglutide and 250 mg sodium caprate.

Group 2 (Eudragit L100 coated reference capsules):

Enteric coated (reference) capsules from Example 8 were filled with a homogenous powder blend containing 10 mg semaglutide and 250 mg sodium caprate.

After oral administration, blood samples were collected at 2, 4, 6, 8, and 24 hours timepoints with 2 ml/timepoint. Serum samples were prepared and analyzed via ELISA (Semaglutide Peninsula Laboratories International, Inc. Cat. No. S-1530).

Results:

In group 1, a delayed onset of semaglutide plasma concentration increase was seen. Average Tₘₐₓ in this group was 8 hours, compared with an average Tₘₐₓ of 4 hours in group 2. The average AUC₀₋₂₄ in group 1 (n=3) was 14-fold higher than in group 2 (n=4).

TABLE 1

| | | | | Avg. | AUC |
|---|---|---|---|---|---|
| | | Tₘₐₓ | Cₘₐₓ | AUC₀₋₂₄ | (improvement |
| | Coating | (hours) | (ng/ml) | (ng/ml*min) | factor) |
| Group 1 | Eudragit | 8 | 56 | 41574 | 14 |
| | NM30D/ | | | | |
| | L30D-55 + | | | | |
| | L100 top | | | | |
| | coating | | | | |
| Group 2 | Eudragit | 4 | 14 | 2970 | 1 |
| | L100 | | | | |

Conclusion:

A solid oral dosage form coated with a combination of Eudragit NM30D/Eudragit L30 D-55 and a Eudragit L100 top coating improved the oral bioavailability of a GLP-1 agonist 14-fold in comparison with an enteric capsule coated with Eudragit L100.

### Example 12: Enteric Coating of Tablets Comprising Semaglutide and Sodium Caprate with Eudragit NM30D (80%) and Eudragit L30 D-55 (20%)

Preparation of the tablets: A homogenous powder blend with 208 mg of semaglutide, 5.500 g of sodium caprate, 2.420 g of sorbitol, 1.430 g of Avicel PH-101 and 121 mg of magnesium stearate was prepared. Mixing was first done in a mortar followed by mixing with a Topitec powder blender. Aliquots of 880 mg were compressed into tablets with a Korsch EK0 single punch tablet press with an average compression force of approximately 15 kN.

Preparation of the Eudragit dispersion: The pH of 107 g of Eudragit NM 30 D was adjusted to about 3 with 1.3 ml of a 20% (w/w) citric acid solution. 67 g of distilled water were added under conventional stirring. 28 g of Eudragit L 30 D-55 was added followed by the addition of 2 g of Plasacryl T20. The final composition was stirred for 15 minutes with a conventional stirrer.

Coating of tablets: Tablets were blended with placebo tablets of comparable weight and slightly different dimensions (to allow separation after the coating process) and coated with the Eudragit dispersion obtained above using a Glatt GC1 lab coater to final weight between 899 and 909 mg which relates to a coating weight gain between 19 and 29 mg per tablet or 2.8% (w/w). This relates to between 15 mg to 23 mg of Eudragit NM30D (dry substance) per tablet.

### Example 13: Coating of HPMC Capsules with a Combination of 50% Eudragit NM 30 D and 50% Eudragit L 30 D-55

Preparation of the Eudragit dispersions: The pH of 67.5 g of Eudragit NM 30 D was adjusted to about 3 with 1.0 ml of a 20% (w/w) citric acid solution. 67 g of distilled water were added under conventional stirring. 67.5 g of Eudragit L 30 D-55 were then added, followed by the addition of 2 g of Plasacryl T20. The final composition was stirred for 15 minutes with a conventional stirrer.

Coating of HPMC capsules: Empty size 1 HPMC capsules, each weighing about 70 mg, were coated with the Eudragit dispersion using a Glatt GC1 lab coater to a final weight of 102 mg (weight gain of 32 mg per capsule). This relates to about 16 mg of Eudragit NM30D (dry substance) per size 1 capsule or 3.9 mg Eudragit NM30D (dry substance) per capsule surface cm² (calculated with an average surface area of 4.1 cm² for a hard capsule size 1).

### Example 14: Preparation of Exenatide Tablets Comprising Sodium Caprate

Preparation of the tablets: A homogenous powder blend with 60 mg of exenatide, 3.000 g of sodium caprate, 1.320 g of sorbitol, 780 mg of Avicel PH-101 and 60 mg of magnesium stearate was prepared. Mixing was first done in a mortar followed by mixing with a Topitec powder blender. Aliquots of 870 mg were compressed into tablets with a Korsch EK0 single punch tablet press with an average compression force of approximately 14 kN.

### Example 15: Coating of HPMC Capsules and Exenatide Tablet with a Combination of 65% Eudragit NM 30 D and 35% Eudragit L 30 D-55

Preparation of the Eudragit dispersions: The pH of 87.75 g of Eudragit NM 30 D was adjusted to about 3 with 1.2 ml of a 20% (w/w) citric acid solution. 67 g of distilled water were added under conventional stirring. 47.25 g of Eudragit L 30 D-55 were then added, followed by the addition of 2 g of Plasacryl T20. The final composition was stirred for 15 minutes with a conventional stirrer.

Coating of HPMC capsules: Empty size 1 HPMC capsules, each weighing about 70 mg, and one exenatide tablet from example 14 were coated with the Eudragit dispersion using a Glatt GC1 lab coater to a final capsule weight of 105 mg (weight gain of 35 mg per capsule). This relates to about 23 mg of Eudragit NM30D per size 1 capsule or 5.6 mg Eudragit NM30D per capsule surface cm² (calculated with an average surface area of 4.1 cm² for a hard capsule size 1). The final weight of the exenatide tablet was 941 mg which relates to a weight gain of 71 mg per tablet which further relates to about 46 mg of Eudragit NM30D (dry substance) per tablet.

### Example 16: Coating of HPMC Capsules and Exenatide Tablet with Eudragit L 30 D-55 (Reference Capsules)

Preparation of the Eudragit dispersions: 56.9 g of distilled water were added under conventional stirring to 114 g of Eudragit L 30 D-55, followed by the addition of 29.1 g of Plasacryl T20. The final composition was stirred for 15 minutes with a conventional stirrer.

Coating of HPMC capsules: Empty size 1 HPMC capsules, each weighing about 70 mg, and one exenatide tablet from example 14 were coated with the Eudragit dispersion using a Glatt GC1 lab coater to a final capsule weight of 104 mg (weight gain of 34 mg per capsule). The final weight of the exenatide tablet was 916 mg which relates to a weight gain of 46 mg per tablet.

### Example 17: Coating of HPMC Capsules with 100% Eudragit NM30D (Reference Capsules)

Preparation of the Eudragit dispersions: 114 g of Eudragit NM30D and 2 g of Plasacryl T20 were stirred for 15 minutes with a conventional stirrer.

Coating of HPMC capsules: Empty size 1 HPMC capsules, each weighing about 70 mg, were coated with the Eudragit dispersion using a Glatt GC1 lab coater to a final capsule weight of 106 mg (weight gain of 36 mg per capsule) relating to about 36 mg Eudragit NM30D (dry substance) per capsule or 8.8 mg Eudragit NM30D (dry substance) per capsule surface cm².

### Example 18: Coating of HPMC Capsules and Exenatide Tablet with a Combination of 70% Eudragit NM 30 D and 30% Eudragit L 30 D-55

Preparation of the Eudragit dispersions: The pH of 94.5 g of Eudragit NM 30 D was adjusted to about 3 with 1.2 ml of a 20% (w/w) citric acid solution. 67 g of distilled water were added under conventional stirring. 40.5 g of Eudragit L 30 D-55 were then added, followed by the addition of 2 g of Plasacryl T20. The final composition was stirred for 15 minutes with a conventional stirrer.

Coating of HPMC capsules: Empty size 1 HPMC capsules, each weighing about 70 mg, and one exenatide tablet from example 14 were coated with the Eudragit dispersion using a Glatt GC1 lab coater to a final capsule weight of 104 mg (weight gain of 34 mg per capsule). This relates to about 24 mg of Eudragit NM30D per size 1 capsule or 5.9 mg Eudragit NM30D per capsule surface cm². The final weight of the exenatide tablet was 907 mg which relates to a weight gain of 37 mg per tablet which further relates to about 26 mg of Eudragit NM30D (dry substance) per tablet.

### Example 19: Coating of HPMC Capsules and Exenatide Tablet with Eudragit FL 30 D-55

Preparation of the Eudragit dispersions: 30 g of talc were suspended in 370 g of H₂O and homogenized with an Ultra Turrax for 10 minutes. 200 g of Eudragit FL 30 D-55 were then added. The final composition was stirred for 15 minutes with a conventional stirrer.

Coating of HPMC capsules: Empty size 1 HPMC capsules, each weighing about 70 mg, and one exenatide tablet from example 14 were coated with the Eudragit dispersion using a Glatt GC1 lab coater to a final capsule weight gains of 24, 31 and 46% which relates to 16.8, 21.7 and 32.2 mg coating weight gain per capsule, respectively, calculated on the weight of the empty capsules which further relates to 10.2, 13.2 and 19.6 mg Eudragit NM30D per size 1 capsule or 2.5, 3.2 and 4.8 mg Eudragit NM30D (dry substance) per capsule surface cm². The final weight of the coated exenatide tablet was 905 mg which relates to a weight gain of 35 mg per tablet.

### Example 20: Preparation of Tablets Comprising Semaglutide and Sodium Caprate

Preparation of the tablets: A homogenous powder blend with 146.4 mg of semaglutide, 4.125 g of sodium caprate, 1.815 g of sorbitol, 1.072 g of Avicel PH-101 and 99 mg of magnesium stearate was prepared. Mixing was first done in a mortar followed by mixing with a Topitec powder blender. Aliquots of 880 mg were compressed into tablets with a Korsch EK0 single punch tablet press with an average compression force of approximately 15 kN.

### Example 21: Enteric Coating of Tablets Comprising Semaglutide and Sodium Caprate with Eudragit NM30D (80%) and Eudragit L30 D-55 (20%)

Preparation of the Eudragit dispersion: The pH of 107 g of Eudragit NM 30 D was adjusted to about 3 with 1.3 ml of a 20% (w/w) citric acid solution. 67 g of distilled water were added under conventional stirring. 28 g of Eudragit L 30 D-55 was added followed by the addition of 2 g of Plasacryl T20. The final composition was stirred for 15 minutes with a conventional stirrer.

Coating of tablets: 4 Tablets (from example 20) were blended with placebo tablets of comparable weight and slightly different dimensions (to allow separation after the coating process) and coated with the Eudragit dispersion obtained above using a Glatt GC1 lab coater to final weight of about 965 mg which relates to a coating weight gain of about 85 mg per tablet or 9.7% (w/w). This relates to about 68 mg of Eudragit NM30D (dry substance) per tablet.

### Example 22: Enteric Coating of Tablets Comprising Semaglutide and Sodium Caprate with Eudragit NM30D (50%) and Eudragit L30 D-55 (50%)

Preparation of the Eudragit dispersion: The pH of 67 g of Eudragit NM 30 D was adjusted to about 3 with 1.0 ml of a 20% (w/w) citric acid solution. 67 g of distilled water were added under conventional stirring. 67 g of Eudragit L 30 D-55 was added followed by the addition of 2 g of Plasacryl T20. The final composition was stirred for 15 minutes with a conventional stirrer.

Coating of tablets: 4 Tablets (from example 20) were blended with placebo tablets of comparable weight and slightly different dimensions (to allow separation after the coating process) and coated with the Eudragit dispersion obtained above using a Glatt GC1 lab coater to final weight of about 910 mg which relates to a coating weight gain of about 25 mg per tablet. This relates to about 12.5 mg of Eudragit NM30D (dry substance) per tablet.

### Example 23: Preparation of Tablets Comprising Semaglutide and Sodium Caprate

Preparation of the tablets: A homogenous powder blend with 220 mg of semaglutide, 5.500 g of sodium caprate, 2.420 g of sorbitol, 1.430 g of Avicel PH-101 and 110 mg of magnesium stearate was prepared. Mixing was first done in a mortar followed by mixing with a Topitec powder blender. Aliquots of 880 mg were compressed into tablets with a Korsch EK0 single punch tablet press with an average compression force of approximately 15 kN.

### Example 24: Enteric Coating of Tablets Comprising Semaglutide and Sodium Caprate with Eudragit NM30D (80%) and Eudragit L30 D-55 (20%)

Preparation of the Eudragit dispersion: The pH of 160.5 g of Eudragit NM 30 D was adjusted to about 3 with 2.0 ml of a 20% (w/w) citric acid solution. 100.5 g of distilled water were added under conventional stirring. 42 g of Eudragit L 30 D-55 was added followed by the addition of 3 g of Plasacryl T20. The final composition was stirred for 15 minutes with a conventional stirrer.

Coating of tablets: 10 Tablets (from example 23) were blended with placebo tablets of comparable weight and slightly different dimensions (to allow separation after the coating process) and coated with the Eudragit dispersion obtained above using a Glatt GC1 lab coater to final weight of about 1008 mg which relates to a coating weight gain of about 128 mg per tablet (about 14.6% weight gain). This relates to about 102.4 mg of Eudragit NM30D (dry substance) per tablet.

### Example 25: Enteric Coating of Capsules and Tablets Comprising Semaglutide and Sodium Caprate with Eudragit NM30D (80%) and Eudragit FS30D (20%)

Preparation of the Eudragit dispersion: The pH of 160.5 g of Eudragit NM 30 D was adjusted to about 3 with 2.0 ml of a 20% (w/w) citric acid solution. 100.5 g of distilled water were added under conventional stirring. 42 g of Eudragit FS30D was added followed by the addition of 3 g of Plasacryl T20. The final composition was stirred for 15 minutes with a conventional stirrer.

Coating of capsules and one tablet: 1 Tablet (from example 14) was blended with empty size 1 HPMC capsules and coated with the Eudragit dispersion obtained above using a Glatt GC1 lab coater to final capsule weight of about 102 mg which relates to a coating weight gain of about 32 mg per size 1 capsule. The tablet was coated to a final weight of 909 mg which relates to a weight gain of 4.5% (w/w).

### Example 26: Dissolution Testing of Coated Capsules

Pre-coated capsules from example 16, example 13, example 15, example 2, example 1 and example 17 were filled each with 10 mg semaglutide and 100 mg of sodium caprate. Dissolution studies were performed with an Erweka DT light 126, using the basket method at 37° C. and a rotation speed of 75 rpm. A modified version of the United States Pharmacopeia (USP) method was used. The capsules were put into the baskets and the baskets were placed in open blue cap bottles, containing 100 ml of the according dissolution media. The blue cap bottles were in direct contact with water, ensuring a core temperature inside the blue cap bottles of 37° C. (temperature was confirmed prior to starting the test with an external thermometer). Baskets were first placed into simulated gastric fluid according to USP for one hour, then for 3 hours in simulated intestinal fluid (SIF) according to USP with pH 6.0. Samples of 1 ml were withdrawn after 60 minutes and at further pre-determined time-points. 40 μl of the samples were injected into a HPLC system using a reversed phase gradient method (water/acetonitrile+0.1% trifluoracetic acid; column: Waters Xselect CSH C18). Sampling was performed until full capsule dissolution, defined by a semaglutide release of >75% of the theoretical release value.

The results thus obtained are shown in FIG. 2. These results show that all capsules were stable at the acid stage but only capsules coated with increasing amounts of Eudragit NM30D demonstrate afterwards a lag time at intestinal pH 6 levels followed by a rather fast release profile. Only the capsule coated with 100% Eudragit NM30D did not dissolve within 3 hours at pH 6.

### Example 27: Dissolution Testing of Capsules Coated with Eudragit FL 30 D-55

Pre-coated capsules from example 19 were each filled with 10 mg semaglutide and 100 mg of sodium caprate. Dissolution studies were performed as described in Example 26.

The results thus obtained are shown in FIG. 3. These results show increasing weight gains of Eudragit FL 30 D-55 change the release profile of semaglutide. Typical weight gains did not result in significantly different release profiles of GLP-1 agonists but a weight gain of 46% calculated on the empty capsule which relates to 32.2 mg coating weight gain per size 1 capsule resulted in a dissolution lag time of at least 40 minutes at pH 6.

### Example 28: Dissolution of GLP-1 Agonist Peptides from Capsules Coated with Eudragit L30D-55 (20%) and Eudragit NM30D (80%)

Pre-coated capsules from example 1 were filled with a) 5 mg Exenatide and 120 mg of sodium caprate, b) 5 mg Semaglutide and 120 mg sodium caprate, c) 5 mg Tirzepatide and 120 mg of sodium caprate. Dissolution studies were performed as described in Example 26.

The results thus obtained are shown in FIG. 4. These results show that enteric dosage forms comprising different GLP-1 agonists demonstrate comparable dissolution profiles with a dissolution lag time of at least 90 minutes at pH 6 followed by a relatively rapid dissolution.

### Example 29: Stability of Enteric Solid Dosage Forms Comprising GLP-1 Agonists in FEDGAS

The aim of this experiment is to demonstrate the robustness of coated solid dosage forms according to the invention in simulated fed state gastric media. Pre-coated capsules and exenatide tablets from examples 1, 4, 13, 15, 16, 19 and 25 were used and in case of capsules filled with 10 mg exenatide and 200 mg sodium caprate. Dissolution testing was done in FEDGAS (Fed State Simulated Gastric Fluid) at pH 6. FEDGAS™ pH 6 was prepared according to protocols from Biorelevant (www.biorelevant.com). For each 100 ml of final media, 4.1 ml of Biorelevant buffer pH 6, 81.4 g of water and 17 g of FEDGAS gel were mixed together. Dissolution studies were performed with an Erweka DT light 126, using the basket method at 37° C. and a rotation speed of 75 rpm. A modified version of the United States Pharmacopeia (USP) method was used. The capsules were put into the baskets and the baskets were placed in open blue cap bottles, containing 100 ml of the FEDGAS pH 6 media. The blue cap bottles were in direct contact with water, ensuring a core temperature inside the blue cap bottles of 37° C. (temperature was confirmed prior to starting the test with an external thermometer). Baskets were placed into FEDGAS pH 6 media for 4 hours. Samples of 1 ml were withdrawn after 60 minutes and at further pre-determined time-points. 40 μl of the samples were injected into a HPLC system using a reversed phase gradient method (water/acetonitrile+0.1% trifluoracetic acid; column: Waters Xselect CSH C18).

The results thus obtained are shown in FIGS. 5, 6 and 7. These results show that:
Increasing amounts of Eudragit NM30D increase the stability of solid dosage forms comprising GLP-1 peptides in simulated fed state.
Coated tablets according to the invention are more robust in simulated fed state than coated capsules.
Coated capsules and tablets as described in example 25 are most robust in simulated fed state.

### Example 30: Coating of Capsules with Acryl EZE and Opadry EC/Acryl EZE

HPMC capsules of size 4 were enteric coated either only with Acryl-EZE or with two coating layers of Opadry EC to a weight gain of 16% (w/w) as a first layer followed by additional Acryl-EZE to a weight gain of 32% (w/w) as second coating layer. These capsules were then filled with 5 mg of Semaglutide and 80 mg of Sodium Caprate and tested for their stability in FEDGAS pH 6 media according to the method as described in Example 29. The results are shown in FIG. 8. Results show that the combination of Opadry EC and Acryl EZE are much more robust in simulated fed state than capsules only coated with Acryl EZE.

### Example 31: Oral Administration of Coated Solid Oral Dosage Forms Comprising Semaglutide in Beagle Dogs

Coated capsules from Examples 1 and 16 were filled with 10 mg Semaglutide and 250 mg Sodium Caprate. Further tablets from Examples 22 and 24 were used for oral administration to dogs. The dogs were fasted overnight for at least 12 hours. In the morning respective capsules or tablets were administered (one tablet or capsule per dog) with 10 ml of water. After administration blood samples were collected at time points 0, 1, 2, 4, 8, 12, 24 and 48 hours and kept frozen until analysis. Blood samples were analyzed with a commercial Semaglutide ELISA Kit.

The results thus obtained are shown in FIG. 9 and Table 2. These results show that:
The addition of Eudragit NM30D considerably increases the oral bioavailability compared with classical enteric coatings without Eudragit NM30D.
Increasing amounts of Eudragit NM30D further increase the oral bioavailability of solid dosage forms comprising GLP-1 peptides.
Coated tablets according to the invention further increase the oral bioavailability GLP-1 peptides compared to coated capsules.

TABLE 2
Pharmacokinetic parameters and improvement factors
of semaglutide enteric capsules after oral dosing
to beagle dogs in fasted stage (n = 4)

| | | Avg. Cₘₐₓ | Avg. AUC_{(0-24 h)} |
|---|---|---|---|
| | | improvement | improvement |
| Composition | Tₘₐₓ | factor | factor |
| 10 mg Semaglutide, 250 mg | 2 hours | 1 | 1 |
| Sodium Caprate in capsule | | | |
| from Example 16 | | | |
| 10 mg Semaglutide, 250 mg | 6 hours | 3-fold | 16-fold |
| Sodium Caprate in capsule | | | |
| from Example 1 | | | |

Conclusions: Enteric solid oral dosage forms comprising Eudragit NM30D facilitate significant oral bioavailability of GLP-1 agonists after oral administration. Increasing the amount of Eudragit NM30D further improves the bioavailability of GLP-1 agonists after oral administration. Enteric coating of capsules comprising Eudragit NM30D increased the AUC 16-fold compared to a standard enteric coated capsule without Eudragit NM30D.

### Example 32: Food Interaction after Oral Administration of Semaglutide Tablets to Dogs

Food interaction was evaluated when tablets from Examples 12, 21 and 24 were administered orally after feeding. The presence of food within the stomach may interfere with the performance of orally administered peptide drugs. The dogs were fasted overnight for at least 12 hours. In the morning dogs from the fed-group were given a light meal (Hills I/D). 30 minutes following the diet the tablets were administered (one tablet per dog) with 10 ml of water. After administration blood samples were collected at time points 0, 1, 2, 4, 8, 12, 24 and 48 hours and kept frozen until analysis. Blood samples were analyzed with a commercial Semaglutide ELISA Kit.

The results thus obtained are shown in Tables 3 and 4. These results show that increasing amounts of Eudragit NM30D increase the oral bioavailability of solid dosage forms comprising GLP-1 peptides in fed state.

TABLE 3
AUC improvement as a function of coating weight
gain after oral dosing in fed state (n = 4)

| | Coating weight | Cₘₐₓ improvement |
|---|---|---|
| | gain (% w/w) | factor |
| Tablets from example 12 | 2.8 | 1 |
| Tablets from example 21 | 9.7 | 17 |
| Tablets from example 24 | 14.6 | 27 |

Conclusion: Increasing the weight gain of coated tablets according to the invention improves the bioavailability of GLP-1 agonists in fed state.

TABLE 4
Pharmacokinetic parameters and improvement factors of semaglutide
enteric tablets after oral dosing to beagle dogs in fasted
and fed stage (n = 5). The experiment in the fasted state
was performed as described in Example 31. The experiment in
the fed state was performed as described in Example 32.

| | AUC₍₀₋₄₈₎ | Cₘₐₓ | C_{48 h} |
|---|---|---|---|
| | improvement | improvement | improvement |
| | factor | factor | factor |
| Oral dosing of tablets from | 1 | 1 | 1 |
| example 24 in the fasted state | | | |
| Oral dosing of tablets from | 0.75 | 1.2 | 2.4 |
| example 24 in the fed state | | | |

Conclusion: A lower onset and thus a later Tₘₐₓ was observed in the fed state most likely due to delayed gastric emptying. An AUC of at least 75% compared to the fasted state was achieved. Due to these PK characteristics and taking into account the long plasma half-life of semaglutide, an AUC in the fed state of >75% compared to the fasted state could be achieved.

1. A solid oral pharmaceutical composition comprising:
(i) a core comprising a GLP-1 receptor agonist, and
(ii) a first coating, wherein the first coating comprises
(ii-1) a copolymer (A) in combination with
(ii-2) a copolymer (B) and/or a copolymer (C) and/or a copolymer (D);
wherein the copolymer (A) comprises:
(a) 20 to 90 mol-% ethyl acrylate repeating units, and
(b) 10 to 80 mol-% methyl methacrylate repeating units;
wherein the copolymer (B), if present, comprises:
(a) 25 to 75 mol-% methacrylic acid repeating units, and
(b) 25 to 75 mol-% ethyl acrylate repeating units;
wherein the copolymer (C), if present, comprises:
(a) 25 to 60 mol-% methacrylic acid repeating units, and
(b) 40 to 75 mol-% methyl methacrylate repeating units;
wherein the copolymer (D), if present, comprises:
(a) 5 to 20 mol-% methacrylic acid repeating units, and
(b) 20 to 40 mol-% methyl methacrylate repeating units, and
(c) 60 to 75 mol-% methyl acrylate repeating units.

2. The solid oral pharmaceutical composition according to claim 1, wherein the first coating comprises
(ii-1) a copolymer (A) in combination with
(ii-2) a copolymer (B) and/or a copolymer (C).

3. The solid oral pharmaceutical composition according to claim 1 or 2, wherein the copolymer (A) in the first coating comprises 60 to 75 mol-% ethyl acrylate repeating units, and 25 to 40 mol-% methyl methacrylate repeating units.

4. The solid oral pharmaceutical composition according to any one of claims 1 to 3, wherein the copolymer (A) in the first coating comprises ethyl acrylate repeating units and methyl methacrylate repeating units in a molar ratio of 2:1.

5. The solid oral pharmaceutical composition according to claim 1 or 2, wherein the copolymer (A) in the first coating further comprises 0.5 to 20 mol-%, preferably 1 to 15 mol-%, 2-(trimethylammonio)ethyl methacrylate chloride repeating units.

6. The solid oral pharmaceutical composition according to any one of claims 1 to 5, wherein the copolymer (B) in the first coating comprises 45 to 55 mol-% methacrylic acid repeating units, and 45 to 55 mol-% ethyl acrylate repeating units.

7. The solid oral pharmaceutical composition according to any one of claims 1 to 6, wherein the copolymer (B) in the first coating comprises methacrylic acid repeating units and ethyl acrylate repeating units in a molar ratio of 1:1.

8. The solid oral pharmaceutical composition according to any one of claims 1 to 7, wherein the copolymer (B) in the first coating consists of methacrylic acid repeating units and ethyl acrylate repeating units.

9. The solid oral pharmaceutical composition according to any one of claims 1 to 8, wherein the first coating comprises the copolymer (A) and the copolymer (B), wherein the content of the copolymer (A) in the first coating is at least 25% (w/w), preferably at least 50% (w/w), more preferably at least 75% (w/w), even more preferably at least 80% (w/w), yet even more preferably at least 90% (w/w), in relation to the total weight of the copolymer (A) and the copolymer (B) in the first coating.

10. The solid oral pharmaceutical composition according to any one of claims 1 to 9, further comprising:
(iii) a second coating which is exterior to the first coating, wherein the second coating comprises a copolymer (C);
wherein the copolymer (C) comprises:
(a) 25 to 60 mol-% methacrylic acid repeating units, and
(b) 40 to 75 mol-% methyl methacrylate repeating units.

11. The solid oral pharmaceutical composition according to any one of claims 1 to 10, wherein the GLP-1 receptor agonist is selected from semaglutide, liraglutide, exenatide, albiglutide, dulaglutide, lixisenatide, taspoglutide, langlenatide, beinaglutide, efpeglenatide, GLP-1(7-37), GLP-1(7-36)NH₂, and oxyntomodulin.

12. The solid oral pharmaceutical composition according to any one of claims 1 to 11, wherein the solid oral pharmaceutical composition is an oral dosage form; preferably wherein the solid oral pharmaceutical composition is in the form of a capsule or a tablet, or wherein the core is in the form of a multiparticulate, a granulate or pellets.

13. The solid oral pharmaceutical composition according to any one of claims 1 to 12, wherein the solid oral pharmaceutical composition has a dissolution profile, as determined by the dissolution method according to USP, with less than 5% of the GLP-1 receptor agonist released within 2 hours in simulated gastric fluid, followed by dissolution in simulated intestinal fluid at pH between 6 and 6.5 with a lag time of at least 1 hour, whereby not more than 10% of the GLP-1 receptor agonist is released within the lag time, and whereby after the lag time more than 75% of the GLP-1 receptor agonist is released in simulated intestinal fluid at pH between 6 and 6.5 within 1 hour.

14. The solid oral pharmaceutical composition according to any one of claims 1 to 13 for use in the treatment or prevention of diabetes, obesity, non-alcoholic fatty liver disease, non-alcoholic steatohepatitis (NASH) or a cardiovascular disease, preferably for use in the treatment or prevention of type 2 diabetes.

15. The solid oral pharmaceutical composition for use according to claim 14, wherein the solid oral pharmaceutical composition is to be administered orally.
